(19) United States
(12) Patent Application Publication
(10) Pub. No.: US 2026/0083323 A1
(43) Pub. Date: Mar. 26, 2026

(54) DRY-EYE PREDICTION

(71) Applicant: Apple Inc., Cupertino, CA (US)

(72) Inventors: Irene CHEN, San Jose, CA (US); Emmanuel B. ALABI, San Diego, CA (US)

(21) Appl. No.: 19/329,226

(22) Filed: Sep. 15, 2025

Related U.S. Application Data
(60) Provisional application No. 63/699,789, filed on Sep. 26, 2024.

(51) Int. Cl. A61B 3/113; A61B 3/10; G06T 7/00; G06V 40/18

(52) U.S. Cl. CPC A61B 3/113; A61B 3/101; G06T 7/0012; G06V 40/197; G06T 2207/30041

(57) ABSTRACT

An electronic device obtains, via the one or more sensors, data including one or more images of one or both eyes. One or more features can be extracted for the one or both eyes of a user of the electronic device. In accordance with a determination that one or more one or more criteria are satisfied, including at least once criterion based on the one or more features of the one or both eyes of the user such as a blinking condition, the electronic device predicts a risk of dry-eye condition associated with the one or both eyes of the user. In some examples, in accordance with a determination that a risk of dry-eye condition is associated with the one or both eyes of the user, the electronic device provides one or more mitigations.

### CROSS REFERENCE TO RELATED APPLICATIONS

This application claims the benefit of U.S. Provisional Application No. 63/699,789, filed Sep. 26, 2024, the content of which is herein incorporated by reference in its entirety for all purposes.

### FIELD OF THE DISCLOSURE

This relates generally to systems and methods for monitoring one or both eyes, and more specifically, to determining a blinking condition including predicting a risk of dry-eye and mitigating a dry-eye condition for a user of an electronic device.

### BACKGROUND OF THE DISCLOSURE

The use of wearable computing devices has increased recently. Some wearable computing devices take images of the eyes using cameras and use the images to track the direction the eyes are looking.

### SUMMARY OF THE DISCLOSURE

Described herein are systems and methods for using sensor data (such as thermal and visible images) of one or both eyes of a user of an electronic device to determine a blinking condition that can predict a dry-eye condition. In some examples, the system and methods can provide one or more mitigations to help improve the detected blinking condition for the purpose of helping the user to avoid a dry-eye condition. The electronic device can use one or more criteria, including at least one criterion that is based on sensor data of the one or both eyes. The satisfaction of the one or more criteria can be used, in some examples, to determine a blinking condition of the user. In some examples, an electronic device (e.g., a head-mounted device) includes one or image sensors that are positioned to image one or both eyes of the user of the electronic device. In some examples, one or more features can be extracted from the sensor data. In one or more examples, in accordance with a determination that one or more criteria are satisfied, the electronic device provides an indication of a risk of dry-eye condition and/or provides one or more dry-eye mitigations to the user of the electronic device.

In one or more examples, as part of the extracted features of the one or both eyes of the user, the electronic device extracts spatial properties of the one or both eyes of the user of the electronic device. In one or more examples, the electronic device segments the extracted features of the one or both eyes of the user to identify various regions and features of the eye. For example, one or more regions of the eye may be segmented based on the temperature (e.g., within a threshold margin of variance in temperature). In one or more examples, a cooling rate is determined for each temperature region. In one or more examples, the electronic device determines a blinking condition of the one or both eyes of the user and uses the blinking condition to satisfy one or more criteria. In one or more examples, when the electronic device determines that the one or more criteria are satisfied, the electronic device predicts a risk of a dry-eye condition associated with the one or both eyes of the user of the electronic device. In one or more examples, the electronic device notifies the user of a possible dry-eye condition and/or suggests mitigations or follow-up for diagnosis with a health care provider. In one or more examples, the electronic device utilizes fan speed and screen brightness to modify blinking behavior of the user, including inducing blinking of the user to prevent or mitigate a possible dry-eye condition.

The full descriptions of these examples are provided in the Drawings and the Detailed Description, and it is understood that this Summary does not limit the scope of the disclosure in any way.

### BRIEF DESCRIPTION OF THE DRAWINGS

For improved understanding of the various examples described herein, reference should be made to the Detailed Description below along with the following drawings. Like reference numerals often refer to corresponding parts throughout the drawings.

FIG. 1 illustrates an electronic device presenting an extended reality environment according to some examples of the disclosure.

FIG. 2 illustrates a block diagram of an example architecture for a device according to some examples of the disclosure.

FIG. 3 illustrates an example electronic device configured to predict a risk of and prevent and/or mitigate a dry-eye condition of one or both eyes of the user according to some examples of the disclosure.

FIG. 4 is a flow diagram illustrating a method for determining a blinking condition and providing mitigations according to some examples of the disclosure.

FIG. 5 is a flow diagram illustrating a method for predicting a risk of dry-eye using blink completeness and/or blink frequency according to some examples of the disclosure.

FIG. 6 illustrates a representation of features extracted from an open eye and a closed eye of a user of an electronic device according to some examples of the disclosure.

FIG. 7 is an example plot illustrating blinking states of one or both eyes of a user of an electronic device by eye openness and time according to some examples of the disclosure.

FIG. 8 illustrates a representation of corneal glint features extracted from an eye of a user of an electronic device according to some examples of the disclosure.

FIG. 9 illustrates a representation of iris features at various states of eye openness extracted from an eye of a user of an electronic device according to some examples of the disclosure.

FIG. 10 is a flow diagram illustrating a method determining a blinking condition based on extracted corneal glint and iris features according to some examples of the disclosure.

FIG. 11 illustrates a representation of a guided blinking practice notification as a mitigation displayed by an electronic device according to some examples of the disclosure.

FIG. 12 illustrates a representation of an adjusted display brightness of an electronic device according to one or more examples of the disclosure.

FIG. 13 is a flow diagram illustrating a method for adjusting a display brightness of an electronic device as a mitigation according to one or more examples of the disclosure.

FIG. 14 is a flow diagram illustrating a method for adjusting the speed of one or more fans of an electronic device as a mitigation according to one or more examples of the disclosure.

FIG. 15 illustrates a representation of a warning notification displayed by an electronic device according to some examples of the disclosure.

FIG. 16 is a flow diagram illustrating an example process for displaying a notification as a mitigation according to one or more examples of the disclosure.

FIG. 17 illustrates an example method for determining a blinking condition to predict a risk of dry-eye according to examples of the disclosure.

### DETAILED DESCRIPTION

Described herein are systems and methods for using sensor data (such as thermal and visible images) of one or both eyes of a user of an electronic device to determine a blinking condition that can predict a risk of a dry-eye condition and mitigating said dry-eye condition. The electronic device can use one or more criteria, including at least one criterion that is based on sensor data of the one or both eyes, including the determination of a blinking condition of the user. The satisfaction of the one or more criteria can be used, in some examples, to predict a risk of a dry-eye condition of the user. In some examples, an electronic device (e.g., a head-mounted device) includes one or more sensors, including one or more image sensors that are positioned to image one or more of the eyes of the user of the electronic device. In some examples, one or more features can be extracted from the sensor data. In one or more examples, in accordance with a determination that one or more criteria are satisfied, the electronic device provides an indication of a risk of dry-eye condition and/or provides one or more dry-eye mitigations to the user of the electronic device.

In one or more examples, as part of the extracted features of the one or both eyes of the user, the electronic device extracts spatial properties of the one or both eyes of the user of the electronic device. In one or more examples, the electronic device segments the extracted features of the one or both eyes of the user to identify various regions and features of the eye. In one or more examples, the electronic device determines a blinking condition of the one or both eyes of the user and uses the blinking condition to satisfy one or more criteria. In one or more examples, when the electronic device determines that the one or more criteria are satisfied, the electronic device predicts a risk of a dry-eye condition associated with the one or both eyes of the user of the electronic device. In one or more examples, the electronic device notifies the user of a possible dry-eye condition and/or suggests mitigations or follow-up for diagnosis with a health care provider. In one or more examples, the electronic device utilizes fans and screen brightness to modify blinking behavior of the user, including inducing blinking of the user to prevent or mitigate a possible dry-eye condition.

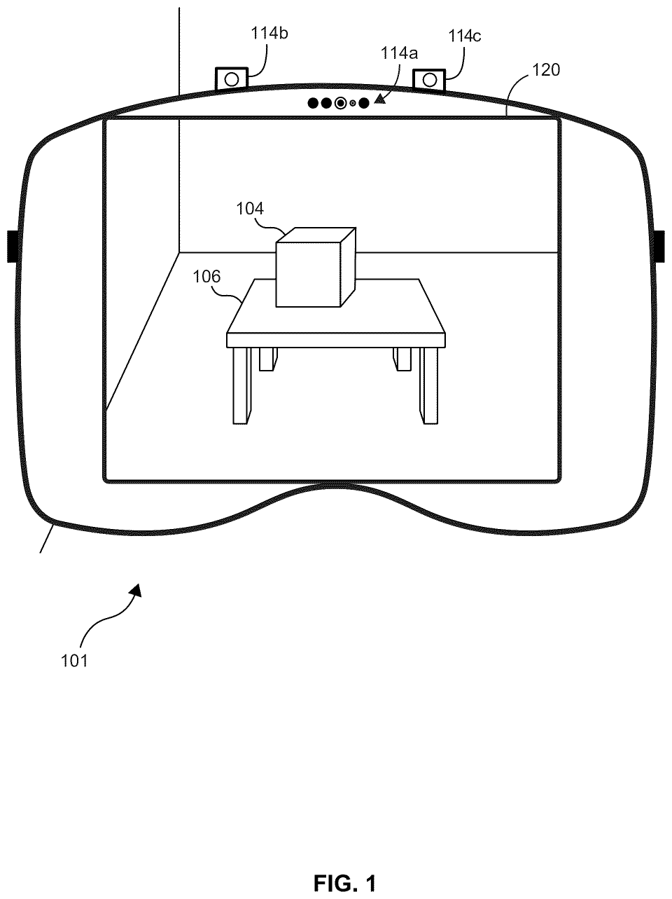

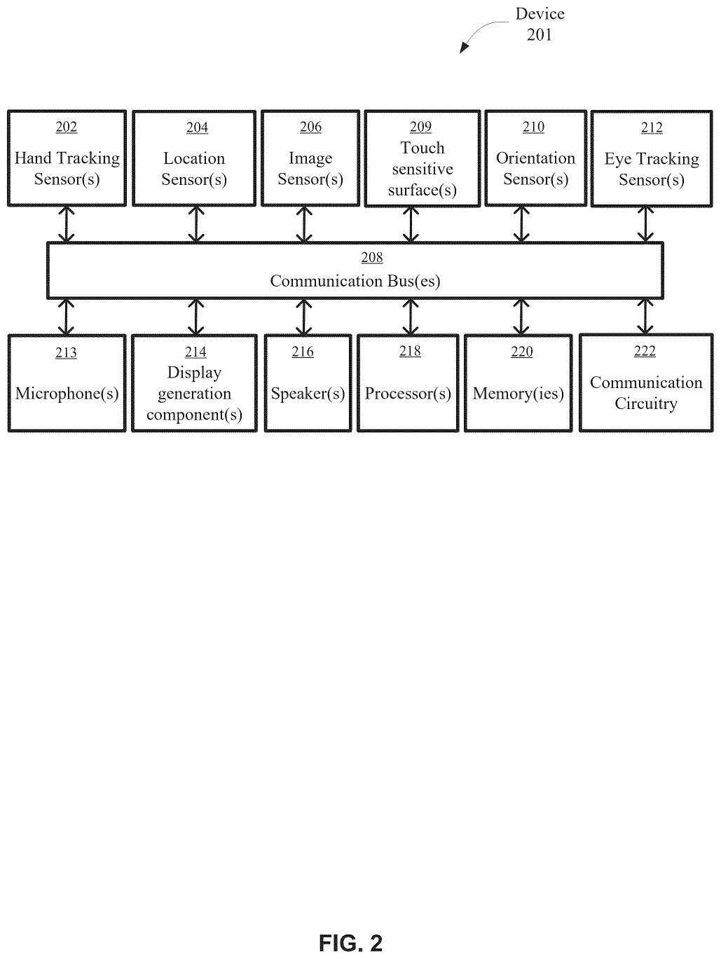

FIG. 1 illustrates an electronic device 101 presenting an extended reality (XR) environment (e.g., a computer-generated environment optionally including representations of physical and/or virtual objects) according to some examples of the disclosure. In some examples, as shown in FIG. 1, electronic device 101 is a head-mounted display or other head-mountable device configured to be worn on a head of a user of the electronic device 101. Examples of electronic device 101 are described below with reference to the architecture block diagram of FIG. 2. As shown in FIG. 1, electronic device 101 and table 106 are located in a physical environment. The physical environment may include physical features such as a physical surface (e.g., floor, walls) or a physical object (e.g., table, lamp, etc.). In some examples, electronic device 101 may be configured to detect and/or capture images of physical environment including table 106 (illustrated in the field of view of electronic device 101).

In some examples, as shown in FIG. 1, electronic device 101 includes one or more internal image sensors 114a oriented towards a face of the user (e.g., eye tracking cameras described below with reference to FIG. 2). In some examples, internal image sensors 114a are used for eye tracking (e.g., detecting a gaze of the user). Internal image sensors 114a are optionally arranged on the left and right portions of display 120 to enable eye tracking of the user's left and right eyes. In some examples, electronic device 101 also includes external image sensors 114b and 114c facing outwards from the user to detect and/or capture the physical environment of the electronic device 101 and/or movements of the user's hands or other body parts.

In some examples, display 120 has a field of view visible to the user (e.g., that may or may not correspond to a field of view of external image sensors 114b and 114c). Because display 120 is optionally part of a head-mounted device, the field of view of display 120 is optionally the same as or similar to the field of view of the user's eyes. In other examples, the field of view of display 120 may be smaller than the field of view of the user's eyes. In some examples, electronic device 101 may be an optical see-through device in which display 120 is a transparent or translucent display through which portions of the physical environment may be directly viewed. In some examples, display 120 may be included within a transparent lens and may overlap all or only a portion of the transparent lens. In other examples, electronic device may be a video-passthrough device in which display 120 is an opaque display configured to display images of the physical environment captured by external image sensors 114b and 114c. While a single display 120 is shown, it should be appreciated that display 120 may include a stereo pair of displays.

In some examples, in response to a trigger, the electronic device 101 may be configured to display a virtual object 104 in the XR environment represented by a cube illustrated in FIG. 1, which is not present in the physical environment, but is displayed in the XR environment positioned on the top of real-world table 106 (or a representation thereof). Optionally, virtual object 104 can be displayed on the surface of the table 106 in the XR environment displayed via the display 120 of the electronic device 101 in response to detecting the planar surface of table 106 in the physical environment 100.

In some examples, the display 120 is provided as a passive component (e.g., rather than an active component) within electronic device 101. For example, the display 120 may be a transparent or translucent display, as mentioned above, and may not be configured to display virtual content (e.g., images of the physical environment captured by external image sensors 114b and 114c and/or virtual object 104). Alternatively, in some examples, the electronic device 101 does not include the display 120. In some such examples in which the display 120 is provided as a passive component or is not included in the electronic device 101, the electronic device 101 may still include sensors (e.g., internal image sensor 114a and/or external image sensors 114b and 114c) and/or other input devices, such as one or more of the components described below with reference to FIG. 2.

It should be understood that virtual object 104 is a representative virtual object and one or more different virtual objects (e.g., of various dimensionality such as two-dimensional or other three-dimensional virtual objects) can be included and rendered in a three-dimensional XR environment. For example, the virtual object can represent an application or a user interface displayed in the XR environment. In some examples, the virtual object can represent content corresponding to the application and/or displayed via the user interface in the XR environment. In some examples, the virtual object 104 is optionally configured to be interactive and responsive to user input (e.g., air gestures, such as air pinch gestures, air tap gestures, and/or air touch gestures), such that a user may virtually touch, tap, move, rotate, or otherwise interact with, the virtual object 104.

In some examples, displaying an object in a three-dimensional environment may include interaction with one or more user interface objects in the three-dimensional environment. For example, initiation of display of the object in the three-dimensional environment can include interaction with one or more virtual options/affordances displayed in the three-dimensional environment. In some examples, a user's gaze may be tracked by the electronic device as an input for identifying one or more virtual options/affordances targeted for selection when initiating display of an object in the three-dimensional environment. For example, gaze can be used to identify one or more virtual options/affordances targeted for selection using another selection input. In some examples, a virtual option/affordance may be selected using hand-tracking input detected via an input device in communication with the electronic device. In some examples, objects displayed in the three-dimensional environment may be moved and/or reoriented in the three-dimensional environment in accordance with movement input detected via the input device.

In the discussion that follows, an electronic device that is in communication with a display generation component and one or more input devices is described. It should be understood that the electronic device optionally is in communication with one or more other physical user-interface devices, such as a touch-sensitive surface, a physical keyboard, a mouse, a joystick, a hand tracking device, an eye tracking device, a stylus, etc. Further, as described above, it should be understood that the described electronic device, display and touch-sensitive surface are optionally distributed amongst two or more devices. Therefore, as used in this disclosure, information displayed on the electronic device or by the electronic device is optionally used to describe information outputted by the electronic device for display on a separate display device (touch-sensitive or not). Similarly, as used in this disclosure, input received on the electronic device (e.g., touch input received on a touch-sensitive surface of the electronic device, or touch input received on the surface of a stylus) is optionally used to describe input received on a separate input device, from which the electronic device receives input information.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, a television channel browsing application, and/or a digital video player application.

FIG. 2 illustrates a block diagram of an example architecture for an electronic device 201 according to some examples of the disclosure. In some examples, electronic device 201 includes one or more electronic devices. For example, the electronic device 201 may be a portable device, an auxiliary device in communication with another device, a head-mounted display, etc., respectively. In some examples, electronic device 201 corresponds to electronic device 101 described above with reference to FIG. 1.

As illustrated in FIG. 2, the electronic device 201 optionally includes various sensors, such as one or more hand tracking sensors 202, one or more location sensors 204, one or more image sensors 206 (optionally corresponding to internal image sensors 114a and/or external image sensors 114b and 114c in FIG. 1), one or more touch-sensitive surfaces 209, one or more motion and/or orientation sensors 210, one or more eye tracking sensors 212, one or more microphones 213 or other audio sensors, one or more body tracking sensors (e.g., torso and/or head tracking sensors), one or more display generation components 214, optionally corresponding to display 120 in FIG. 1, one or more speakers 216, one or more processors 218, one or more memories 220, and/or communication circuitry 222. One or more communication buses 208 are optionally used for communication between the above-mentioned components of electronic devices 201.

Communication circuitry 222 optionally includes circuitry for communicating with electronic devices, networks, such as the Internet, intranets, a wired network and/or a wireless network, cellular networks, and wireless local area networks (LANs). Communication circuitry 222 optionally includes circuitry for communicating using near-field communication (NFC) and/or short-range communication, such as Bluetooth®.

Processor(s) 218 include one or more general processors, one or more graphics processors, and/or one or more digital signal processors. In some examples, memory 220 is a non-transitory computer-readable storage medium (e.g., flash memory, random access memory, or other volatile or non-volatile memory or storage) that stores computer-readable instructions configured to be executed by processor(s) 218 to perform the techniques, processes, and/or methods described below. In some examples, memory 220 can include more than one non-transitory computer-readable storage medium. A non-transitory computer-readable storage medium can be any medium (e.g., excluding a signal) that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on compact disc (CD), digital versatile disc (DVD), or Blu-ray technologies, as well as persistent solid-state memory such as flash, solid-state drives, and the like.

In some examples, display generation component(s) 214 include a single display (e.g., a liquid-crystal display (LCD), organic light-emitting diode (OLED), or other types of display). In some examples, display generation component(s) 214 include multiple displays. In some examples, display generation component(s) 214 can include a display with touch capability (e.g., a touch screen), a projector, a holographic projector, a retinal projector, a transparent or translucent display, etc. In some examples, electronic device 201 includes touch-sensitive surface(s) 209, respectively, for receiving user inputs, such as tap inputs and swipe inputs or other gestures. In some examples, display generation component(s) 214 and touch-sensitive surface(s) 209 form touch-sensitive display(s) (e.g., a touch screen integrated with electronic device 201 or external to electronic device 201 that is in communication with electronic device 201).

Electronic device 201 optionally includes image sensor(s) 206. Image sensors(s) 206 optionally include one or more visible light image sensors, such as charged coupled device (CCD) sensors, and/or complementary metal-oxide-semiconductor (CMOS) sensors operable to obtain images of physical objects from the real-world environment. Image sensor(s) 206 also optionally include one or more infrared (IR) sensors, such as a passive or an active IR sensor, for detecting infrared light from the real-world environment. For example, an active IR sensor includes an IR emitter for emitting infrared light into the real-world environment. Image sensor(s) 206 also optionally include one or more cameras configured to capture movement of physical objects in the real-world environment. Image sensor(s) 206 also optionally include one or more depth sensors configured to detect the distance of physical objects from electronic device 201. In some examples, information from one or more depth sensors can allow the device to identify and differentiate objects in the real-world environment from other objects in the real-world environment. In some examples, one or more depth sensors can allow the device to determine the texture and/or topography of objects in the real-world environment.

In some examples, electronic device 201 uses CCD sensors, event cameras, and depth sensors in combination to detect the physical environment around electronic device 201. In some examples, image sensor(s) 206 include a first image sensor and a second image sensor. The first image sensor and the second image sensor work in tandem and are optionally configured to capture different information of physical objects in the real-world environment. In some examples, the first image sensor is a visible light image sensor and the second image sensor is a depth sensor. In some examples, electronic device 201 uses image sensor(s) 206 to detect the position and orientation of electronic device 201 and/or display generation component(s) 214 in the real-world environment. For example, electronic device 201 uses image sensor(s) 206 to track the position and orientation of display generation component(s) 214 relative to one or more fixed objects in the real-world environment.

In some examples, electronic device 201 includes microphone(s) 213 or other audio sensors. Electronic device 201 optionally uses microphone(s) 213 to detect sound from the user and/or the real-world environment of the user. In some examples, microphone(s) 213 include an array of microphones (a plurality of microphones) that optionally operate in tandem, such as to identify ambient noise or to locate the source of sound in space of the real-world environment.

Electronic device 201 includes location sensor(s) 204 for detecting a location of electronic device 201 and/or display generation component(s) 214. For example, location sensor(s) 204 can include a global positioning system (GPS) receiver that receives data from one or more satellites and allows electronic device 201 to determine the device's absolute position in the physical world.

Electronic device 201 includes orientation sensor(s) 210 for detecting orientation and/or movement of electronic device 201 and/or display generation component(s) 214. For example, electronic device 201 uses orientation sensor(s) 210 to track changes in the position and/or orientation of electronic device 201 and/or display generation component(s) 214, such as with respect to physical objects in the real-world environment. Orientation sensor(s) 210 optionally include one or more gyroscopes and/or one or more accelerometers.

Electronic device 201 includes hand tracking sensor(s) 202 and/or eye tracking sensor(s) 212 (and/or other body tracking sensor(s), such as leg, torso and/or head tracking sensor(s)), in some examples. Hand tracking sensor(s) 202 are configured to track the position/location of one or more portions of the user's hands, and/or motions of one or more portions of the user's hands with respect to the extended reality environment, relative to the display generation component(s) 214, and/or relative to another defined coordinate system. Eye tracking sensor(s) 212 are configured to track the position and movement of a user's gaze (eyes, face, or head, more generally) with respect to the real-world or extended reality environment and/or relative to the display generation component(s) 214. In some examples, hand tracking sensor(s) 202 and/or eye tracking sensor(s) 212 are implemented together with the display generation component(s) 214. In some examples, the hand tracking sensor(s) 202 and/or eye tracking sensor(s) 212 are implemented separate from the display generation component(s) 214.

In some examples, the hand tracking sensor(s) 202 (and/or other body tracking sensor(s), such as leg, torso and/or head tracking sensor(s)) can use image sensor(s) 206 (e.g., one or more IR cameras, 3D cameras, depth cameras, etc.) that capture three-dimensional information from the real-world including one or more body parts (e.g., hands, legs, or torso of a human user). In some examples, the hands can be resolved with sufficient resolution to distinguish fingers and their respective positions. In some examples, one or more image sensors 206 are positioned relative to the user to define a field of view of the image sensor(s) 206 and an interaction space in which finger/hand position, orientation and/or movement captured by the image sensors are used as inputs (e.g., to distinguish from a user's resting hand or other hands of other persons in the real-world environment). Tracking the fingers/hands for input (e.g., gestures, touch, tap, etc.) can be advantageous in that it does not require the user to touch, hold or wear any sort of beacon, sensor, or other marker.

In some examples, eye tracking sensor(s) 212 include at least one eye tracking camera (e.g., infrared (IR) cameras) and/or illumination sources (e.g., IR light sources, such as LEDs) that emit light towards a user's eyes. The eye tracking cameras may be pointed towards a user's eyes to receive reflected IR light from the light sources directly or indirectly from the eyes. In some examples, both eyes are tracked separately by respective eye tracking cameras and illumination sources, and a focus/gaze can be determined from tracking both eyes. In some examples, one eye (e.g., a dominant eye) is tracked by one or more respective eye tracking cameras/illumination sources.

Electronic device 201 is not limited to the components and configuration of FIG. 2, but can include fewer, other, or additional components in multiple configurations. In some examples, electronic device 201 can be implemented between two electronic devices (e.g., as a system). In some such examples, each of the two (or more) electronic device may each include one or more of the same components discussed above, such as various sensors, one or more display generation components, one or more speakers, one or more processors, one or more memories, and/or communication circuitry. A person or persons using electronic device 201, is optionally referred to herein as a user or users of the device.

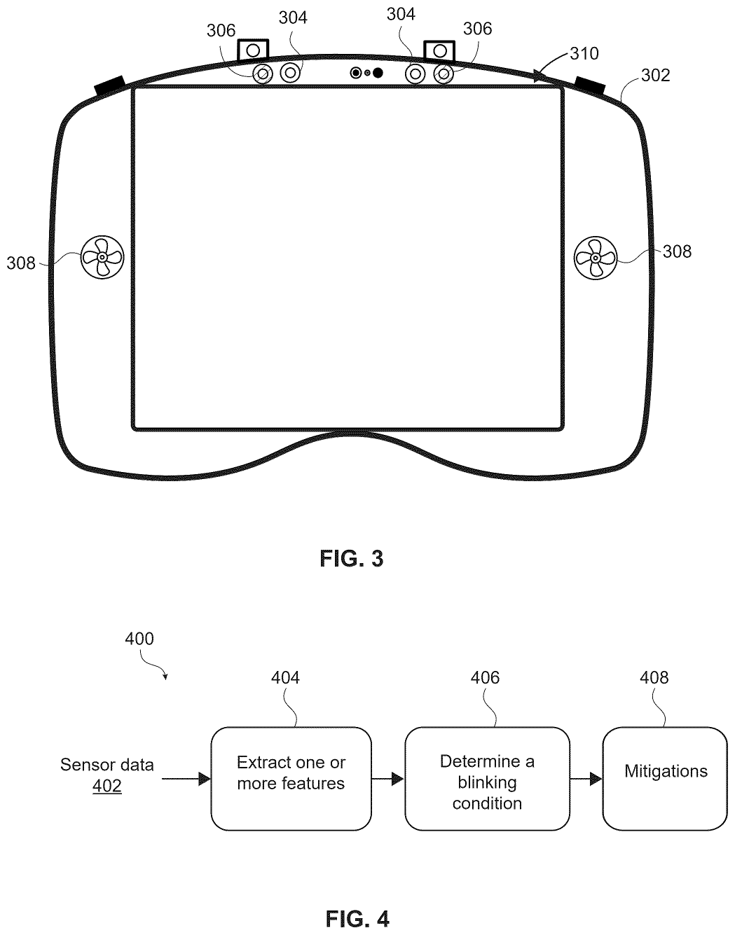

FIG. 3 illustrates an example head mounted device (HMD) 302 for implementing a blink condition detection process according to examples of the disclosure. In the example of FIG. 3, HMD 302 is another example electronic device similar to electronic device 101 described above with respect to FIGS. 1-2 above. In addition to the components of electronic device 101 described above with respect to FIGS. 1-2, in one or more examples, HMD 302 further includes a thermal image sensor 304 and a visible image sensor 306. In one or more examples, thermal image sensor 304 and visible image sensor 306 are part of the image sensors 206 described above with respect to FIG. 2. In one or more examples, both thermal image sensor 304 and visible image sensor 306 are disposed on HMD 302 such that they are directed toward the eyes of the user of the HMD 302. In one or more examples, thermal image sensor 304 is implemented as an infrared sensor that can collect infrared images of the one or both eyes of the user. In one or more examples, visible image sensor 306 is implemented as a camera that collects images of the one or both eyes of the user in the visible light range of wavelengths. As will be described in further detail below, both the thermal image sensor 304 and the visible image sensor 306 (e.g., the data collected by these sensors) can be utilized to detect one or more blinking conditions associated with the eyes of the user.

In one or more examples, the HMD 302 additionally includes one or more fans 308 that are configured, in part, to direct airflow toward the eyes of the user of the HMD 302. In one or more examples, the fans 308 are disposed on HMD 302 such that at least a portion of the airflow generated by the fans 308 will impinge on the one or eyes of the user. As will be discussed in further detail below, the fans 308 can be used as part of one or more mitigations by HMD 302 to relieve a dry-eye condition. In one or more examples, the dry-eye condition is based on a determined blinking condition that is used in predicting the dry-eye condition using the thermal image sensor 304 and visible image sensor 306 data.

In one or more examples, and as described in further detail below, image and thermal data extracted from the eye (using the components described above with respect to FIG. 1) may be used for various purposes related to determining blinking conditions that may be used to predict a risk of dry-eye. As described in detail below, blinking conditions may refer to blinking patterns such as assessing how the eyes of the user of the electronic device open and close, including, blink completeness and blink frequency. For example, in one or more examples, the electronic device can detect when a user may be experiencing irregular blinking, slow blinking, and/or incomplete blinks. In some examples, irregular, slow, and/or incomplete blinks may be a predictor of a dry-eye condition. As described in detail below, dry-eye conditions refer to conditions of the eyes of the user indicating that the eyes are not receiving a sufficient amount of moisture for various reasons (e.g., due to lack of proper blinking, and/or prolonged use of an electronic display). In one or more examples, the systems and methods described below can utilize sensor data, and various processing techniques to extract features from the sensor data that in turn can be used to predict risk of dry-eye conditions (e.g., by detecting a blinking condition). In one or more examples, and as described in further detail below, in response to predicting risk of a dry-eye condition, the electronic device (e.g., HMD 302) can provide one or more mitigations that are configured to mitigate and/or lessen the dry-eye condition.

FIG. 4 illustrates an example blink detection process for detecting blinking conditions and providing mitigations according to one or more examples of the disclosure. In one or more examples, the process 400 of FIG. 4 begins at operation 402 wherein the data collected by thermal image sensor 304 and visible image sensor 306 are received as inputs to process 400. As described above, the data collected by thermal image sensor 304 and visible image sensor 306 include image data from one or both eyes of the user. In one or more examples, after obtaining data from the thermal image sensor 304 and the visible image sensor 306, process 400 moves to operation 404, wherein one or more features are extracted from the data. In one or more examples, and as described in further detail below, the features extracted at operation 404 include (but are not limited to) spatial measurements of the eye, and/or other features associated with the eyes of the user (e.g., blink completeness, blink frequency and other features that are predictive of a dry-eye condition).

In one or more examples, once the features have been extracted from the image sensor data at operation 404, the process moves to operation 406 wherein a blinking condition is determined based on the features extracted from operation 404 of process 400. In one or more examples, and as described in further detail below, a blinking condition can be determined at operation 406 if the one or more extracted features satisfy one or more criteria. For example, operation 406 may detect irregular blinking, which can be used to predict a risk of a dry-eye condition. In one or more examples, once a blinking condition has been determined at operation 406 (thus predicting a dry-eye condition), the process 400 optionally moves to operation 408 wherein the electronic device (e.g., HMD 302) provides one or more mitigations for preventing and treating a dry-eye condition (described in further detail below).

In the example of FIG. 4, inputs from sensor data (e.g., from thermal image sensor 304 and/or visible image sensor 306) are used to extract features and measurements at operation 404 from the eye. The extracted features and measurements at operation 404 may be used to determine a blinking condition at operation 406, and subsequently predict a risk of dry-eye at operation 406 and provide mitigations at operation 408 if appropriate. In one or more examples, extracted features at operation 404 can include thermal, visual, and spatial features of the eyes as the user continues to use the HMD 302. In some examples, as described in further detail below, the extracted features at operation 404 may be used in determining if one or more criteria are satisfied for determining blinking conditions specific to each user.

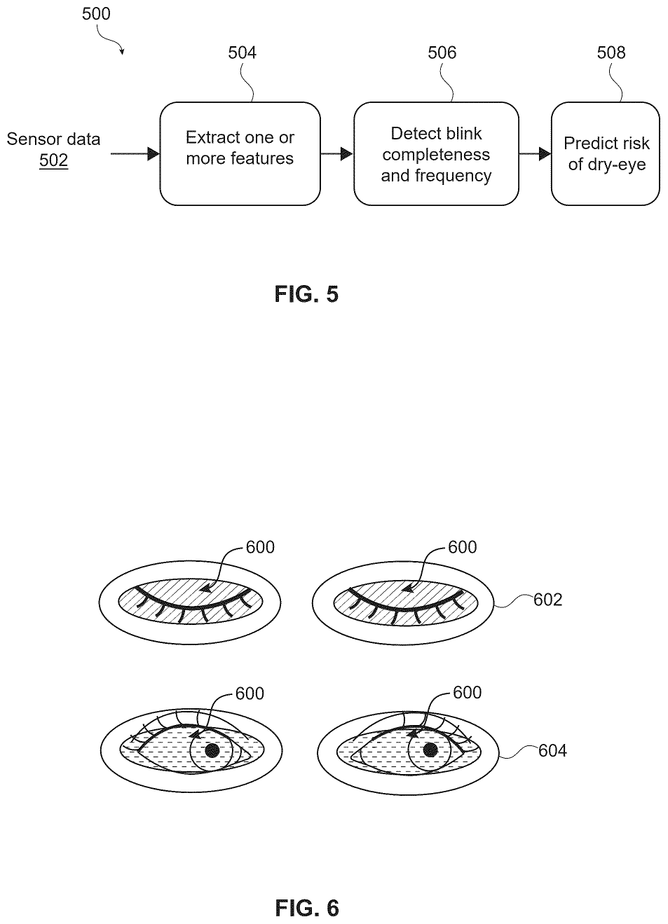

In one or more examples, the features extracted at operation 404 of process 400 include a blink completeness and a blink frequency of one or both eyes of the user of the electronic device. FIG. 5 illustrates an example process for predicting a risk of dry-eye using blink completeness and/or blink frequency according to one or more examples of the disclosure. In one or more examples, the process 500, receives at operation 502 the imaging data from the one or more image sensors described above. In some examples, once the image sensor data has been received at operation 502, the process 500 moves to operation 504 wherein one or more features relating to blink frequency and blink completeness are extracted. For instance, and as described in further detail below, at operation 504, temperature changes in the eye of the user over time can be extracted to detect blink frequency at operation 506, while images of the eye can be analyzed to determine blink completeness of the one or both eyes to predict risk of dry-eye at operation 508 (also described in further detail below).

In one or more examples, once the relevant features are extracted at operation 504, the process 500 moves to operation 506 wherein the blink completeness and the blink frequency are detected based on the features extracted at operation 504. In one or more examples, after the electronic device detects the blink frequency and/or blink completeness at operation 506, the process 500 moves to operation 508 wherein the detected blink frequency and/or the detected blink completeness are compared against one or more pre-determined thresholds to determine if the user of the electronic device has a blinking condition that is predictive of a dry-eye condition. In one or more examples, in response to predicting a risk of a dry-eye at operation 508, operation 408 applies one or more mitigations to prevent or treat the dry-eye condition.

In one or more examples, one of the example features that can be extracted at operation 504 can include a temperature of the eye taken at various times during a blink cycle of the eye of the user. In some examples, detecting variations in temperatures over a plurality of images of an eye taken over a period of time, can be used to detect blink frequency (e.g., the frequency that the user blinks their eyes). FIG. 6 illustrates example states of an eye according to one or more examples of the disclosure. In some examples, and as illustrated in example eye 600 of FIG. 6, blink frequency can include detecting the frequency of changes in the temperature of the surface of the eye from when an eye closes during a blink to a time after the eye opens after the blink has been completed. The example of FIG. 6 illustrates an eye in two states: a closed state 602 and an open state 604. In some examples, an eye that is blinking (e.g., an eye that is closed in the process of a blink) may exhibit an increased temperature when in the closed state 602, specifically around the eyelids, whereas the surface of the eye may decrease in temperature when the eye is in the open state 604. In some examples, an increase in temperature of the surface of the eye 600 during the blink while the eye is in the closed state 602 may be caused by heat generated due to friction from the rubbing of the eyelids. In some examples, a decrease in temperature of the surface of the eye 600 after the opening of the eyes (e.g., when the eyes are in open state 604) may be caused by the evaporation of the tears of the eye. Detecting the frequency at which the eye raises in temperature and subsequently falls in temperature can be used to detect blink frequency. Thus, in one or more examples, blink frequency can be detected from temperature data that is extracted from image data.

In one or more examples, determining a blinking condition (such as blinking frequency) using the change in temperature, may be used to predict a risk of dry-eye in operation 508. In one or more examples, blink completeness can also be used to predict a risk of dry-eye at operation 508. In one or more examples, blink completeness refers to the degree to which the eye of the user shuts during a blink. For instance, in an ideal blink, the eye of the user will shut completely such that the upper eye lid of the user and the lower eye lid of the user contact one another during the blink thereby completely closing obscuring the sclera of the eye of the user.

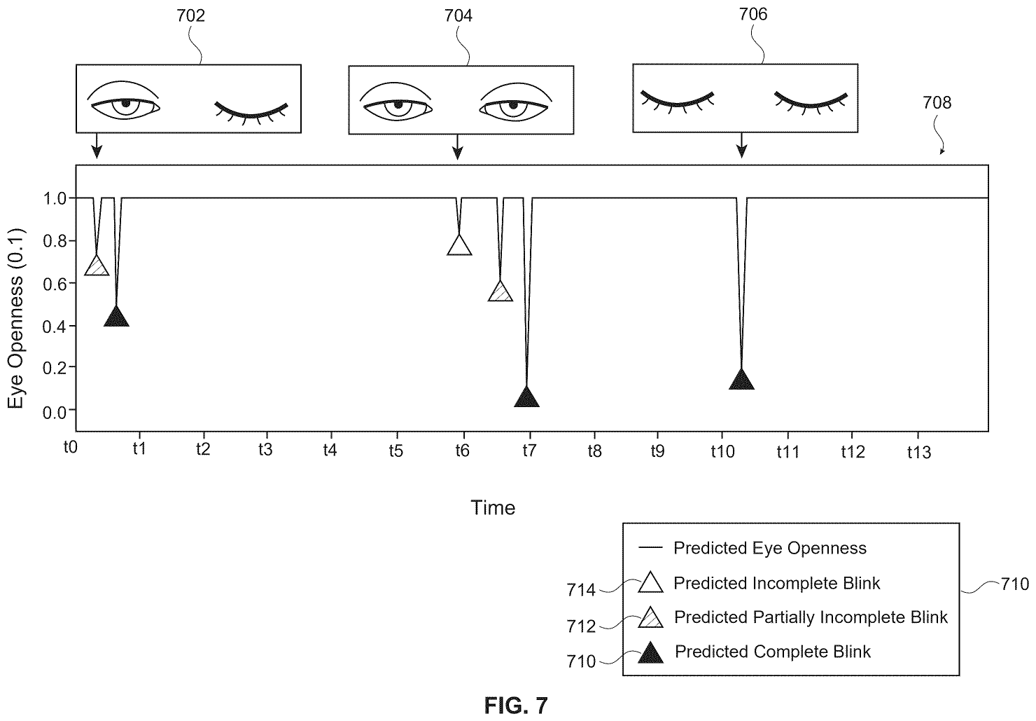

FIG. 7 illustrates an example blink completeness measurement according to one or more examples of the disclosure. In the example of FIG. 7, based on image data obtained of the one or both eyes of the user, electronic device 101 determines an “eye openness” metric of the eyes over a period of time. In one or more examples, the “eye openness” metric provides a quantitative metric that can be used to assess how open the eyes of the user are at any given time. For instance, in one or more examples, an eye openness score of 1.0 indicates that both eyes of the user are fully open, while an eye openness core of 0.0 indicates that both eyes of the user are fully closed (e.g., the upper eye-lid of the user is touching the lower eye-lid of the user).

In one or more examples, eye openness scores between 1.0 and 0.0 can indicate various states of blink completeness. For instance, in one or more examples, the electronic device 101 can determine a “partially incomplete blink” state 702 when one eye of the user is fully closed while the other eye of the user is only partially closed. In some examples, the electronic device 101 can determine an “incomplete blink” state 704 when both eyes of the user do not fully close during a blink. In some examples, the electronic device 101 can detect a “complete blink” state 706, when the electronic device determines that both eyes of the user have completely closed during a blink.

In one or more examples, the electronic device can determine and assign an eye openness score to each blink of user based on a plurality of images of the eyes taken over a period of time (e.g., a minute, an hour, etc.). For instance, in one or more examples, as illustrated by graph 708 which plots the eye openness score of each blink of the user over a period of time, a higher eye openness score 714 can be classified as an “incomplete blink”, a medium eye openness score 712 can be classified as a “partially incomplete blink,” and a lower eye openness score can be classified as “a complete blink,” as indicated at legend 710.

In some examples, if the number and/or frequency of partially incomplete blinks or incomplete blinks are above a threshold number during a given time period, a blinking condition can be determined that is predictive of a dry-eye condition. For example, a number of incomplete blinks above a threshold value over a threshold period of time may be indicative of a risk of dry-eye, as the one or both eyes of the user may not be sufficiently hydrating due to lack of complete blinks.

In one or more examples, the electronic device 101 can determine whether an eye is open, closed, and/or partially open based on the presence of one or more anatomical features of the eye that are extracted at operation 404 of process 400 described above with respect to FIG. 4. As an example, and described herein, the electronic device 101 using the image data collected by the imaging sensors, can look for the presence of corneal glints within the image data to determine the state of openness of an eye.

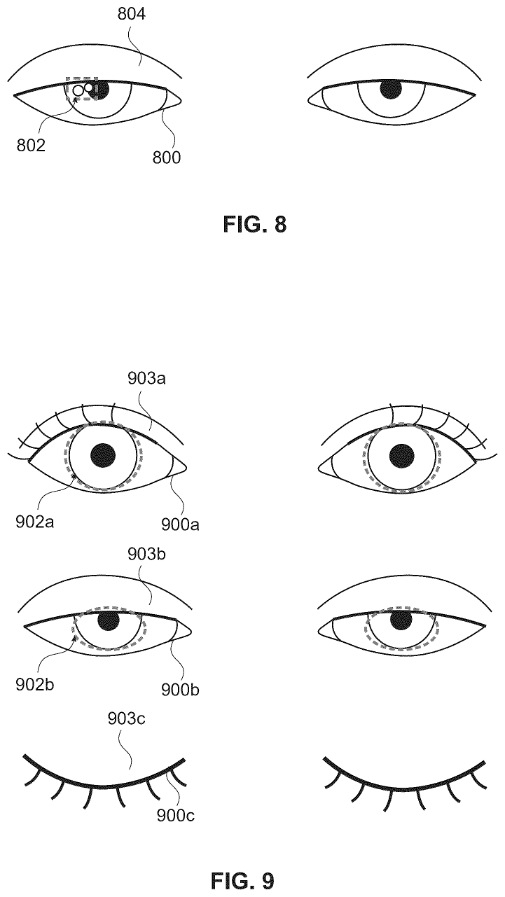

FIG. 8 illustrates an example one or more corneal glints of the eye of the user according to one or more examples of the disclosure. In one or more examples, a corneal glint refers to a reflection of light that is visible in an image of the eye caused by reflections of light reflecting off of the cornea of the user. In one or more examples, the one or more corneal glints 802 of the eye 800 are extracted and processed by one or more machine learning models (e.g., implemented as hardware or using hardware to implement software and/or firmware) that are trained to detect corneal glints from image data of the eyes of the user. In some examples, detecting the presence of one or more corneal glints 802 may indicate the eye 800 is in an open state, as the eyelids 804 are not covering the corneal glints.

In one or more examples, in addition to and/or alternatively to corneal glints, electronic device 101 can extract other anatomical features of the eye such as the iris of the eye to determine blink completeness. FIG. 9 illustrates an example iris of the eye of the user according to one or more examples of the disclosure. In one or more examples, the iris 902a of the eye 900a is extracted and processed by the electronic device 101 to determine the openness state of the eye. In one or more examples, one or more machine learning models (e.g., implemented as hardware or using hardware to implement software and/or firmware) trained to determine the presence of the iris in an image of the eye can be utilized to determine the blink condition of the eye of the user. In one or more examples, detecting the presence of the iris 902a may indicate the eye 900a is in an open state, as the eyelids 903a are not covering iris 902a from being identified by the one or more machine learning models. In one or more examples, the presence of the iris of the eye above a threshold number of images of the eye taken over a period of time may be an indication of an incomplete blink and/or partially incomplete blink. For example, the iris 902b of eye 900b is partially covered by the eyelids 903b, indicating an incomplete blink. In one or more examples, a lack of iris of eye 900c being identified may indicate a closed eye or complete blink. For example, the iris of eye 900c is completely covered by the eyelids 903c, obscuring them from being identified and detected by the one or more machine learning models. In one or more examples, as described in the disclosure, detecting the presence of the iris 902b and iris 902a may further comprise detecting the pupil of one or both eyes to determine a blinking condition. In one or more examples, one or more machine learning models trained to determine the presence of the pupil in an image of the eye can be utilized to determine the blink condition of the eye of the user.

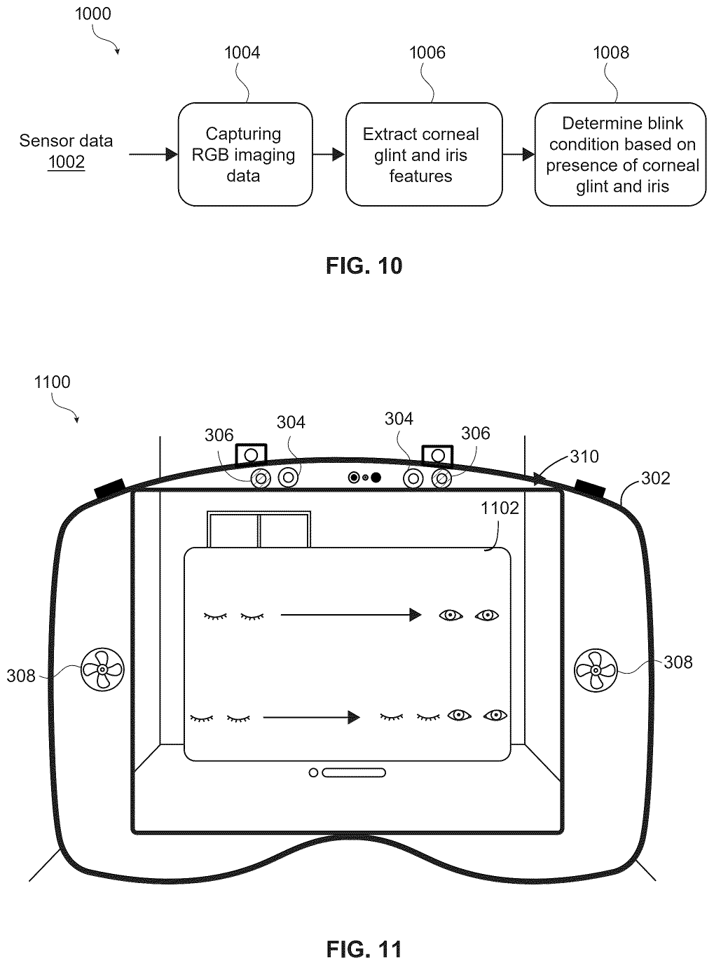

FIG. 10 illustrates an example process for determining a blink condition using visible light data of the eye according to one or more examples of the disclosure. In one or more examples, the process 1000 begins at operation 1004 in which the image sensor data 1002 (and specifically the visible light (RGB) sensor data) is captured at the electronic device. In one or more examples, the image sensor data captured at operation 1004 includes a plurality of images taken of the eye over a period of time (e.g., a minute, an hour, etc.) that includes a pre-determined amount of time after the eye has been determined to have blinked. In one or more examples, once the image sensor data 1002 has been captured at operation 1004, the process 1000 moves to operation 1006 wherein the corneal glint and/or iris features of each image is extracted (according to the methods described above) to determine the blink condition of the eye at a given moment in time at operation 1008. In one or more examples, once the corneal glint and/or iris features are extracted at operation 1006, the process 1000 moves to operation 1008 wherein a blink condition based on the presence of corneal glints and/or iris is determined.

Returning to the example of FIG. 4, once a blinking condition has been determined at operation 406, operation 406 may predict a risk of a dry-eye condition, and the process 400 of FIG. 4 can move to operation 408 wherein one or more mitigations can be provided by the electronic device to mitigate the predicted risk of dry-eye condition. For instance, in one or more examples, a dry-eye condition of the one or both eyes may be caused or worsened by prolonged or improper use of the HMD 302. In such cases, taking a break from use of the device to allow the eyes to rehydrate and return to baseline metrics may be beneficial to the health of the user. In one or more examples, once one or more criteria for predicting a risk of dry-eye at operation 406 is satisfied, the HMD 302 may provide mitigations at operation 408 such as displaying a visual indication (e.g., a notification) to encourage the user to cease use of the device and/or wear the device in a manner that may mitigate the dry-eye condition. In some examples, the visual indication may provide a guided blinking practice to help and instruct the user of the HMD 302 perform regular and complete blinks to mitigate, improve, or prevent a dry-eye condition.

FIG. 11 illustrates an example representation 1100 showing a visual notification displayed by the electronic device to instruct the user on a mitigation for improving, and/or preventing a dry-eye condition according to examples of the disclosure. In one or more examples, the electronic device can display notification 1102 in response to predicting a risk of dry-eye condition. In one or more examples, displaying notification 1102 can include a “guided mitigation” (e.g., an exercise) that can prevent a dry-eye condition. For instance, as an example, the electronic device may display messages such as “In order to prevent dry-eye, close your eyes shut for 3 seconds, followed by opening your eyes for 1 second, followed by closing them again for 3 seconds before opening your eyes again.” In one or more examples, the visual notification 1102 is accompanied by a notification sound, haptic feedback, or any combination thereof that is configured to emphasize the visual notification 1102. In one or more examples, the visual notification 1102 is displayed on the display 310 of the HMD 302 periodically as dry-eye levels are continuously monitored. In one or more examples, the visual notification 1102 includes a selectable button which gives the user an option to close or mute the notification 1102. In one or more examples, the electronic device in addition to providing visual notification 1102 may prevent further use of the HMD 302 without detecting a reduction in current dry-eye levels. In one or more examples, the visual notification 1102 may provide relevant info such as the user's eye metrics described above (e.g., cooling rate), as well as provide historical data on the user's previous determined dry-eye incidents.

In one or more examples, the notification 1102 may further include or further comprise a guided blinking practice. In one or more examples described in the disclosure, guided blinking practice on notification 1102 may include directions on how to practice complete blinks at an appropriate rate. The guided blinking practice on notification 1102 may also present the user's historic eye health information, including information from the one or more extracted features at operation 404 to give the user an understanding of their eye health and dry-eye conditions over time. In one or more examples, the guided blinking practice on notification 1102 is specific to the needs of the user. For example, if the user's blinking frequency is adequate but the user does not blink completely at a rate conducive to eye hydration, the guided blinking practice on notification 1102 may guide the user on how to perform complete blinks. In one or more examples, the guided blinking practice on notification 1102 is part of another application (e.g., a health application) and may be accessed regardless of a risk of a dry-eye condition.

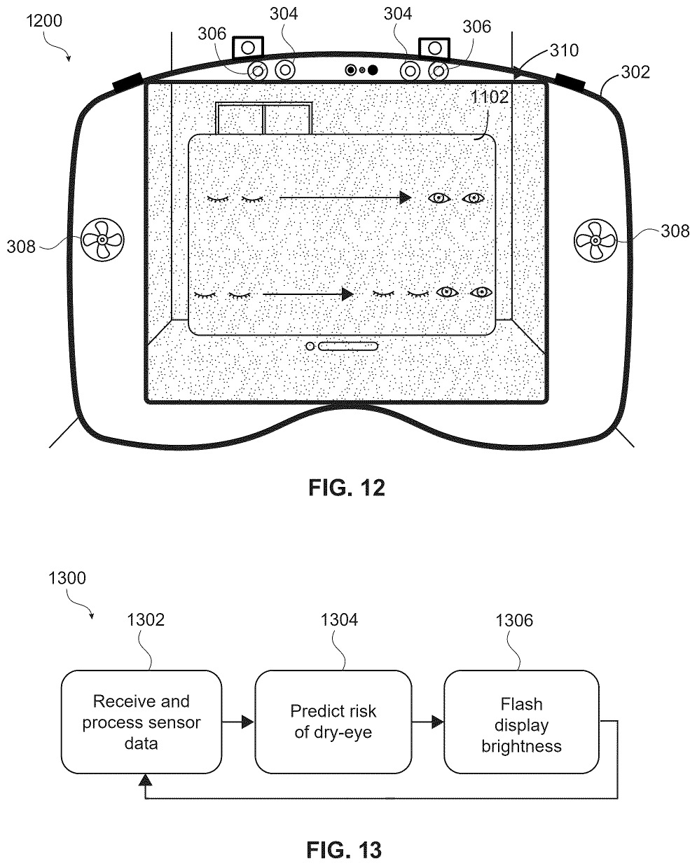

In one or more examples, the HMD 302 may utilize display brightness to modify blinking behavior of the user of the HMD 302. FIG. 12 illustrates an example representation 1200 of HMD 302 modifying blinking behavior of the user, including inducing the user to blink more frequently and/or more completely by increasing the brightness of the display according to one or more examples of the disclosure. The brightness of the display is represented by the shaded region inside the display 310, wherein compared to the display 310 of FIG. 11, FIG. 12 is shaded throughout, illustrating an increase in brightness. For example, rapidly increasing and decreasing the brightness of the display 310 may cause the user to reactively blink due to their eyes not having yet adjusted to the change in brightness. By modifying blinking behavior of the user, including inducing the user to blink after a predicted risk of dry-eye, the predicted dry-eye condition may be avoided before dry-eye happens. In some examples, if the user is already determined to be experiencing dry-eye, inducing them to blink may alleviate their symptoms.

FIG. 13 illustrates an example process for adjusting the display brightness of the device to mitigate and/or prevent a dry-eye condition according to one or more examples of the disclosure. In one or more examples, the process 1300 receives sensor data, and processes the sensor data using one or more machine learning models (e.g., implemented as hardware or using hardware to implement software and/or firmware) at operation 1302. In one or more examples, extracting features at operation 1006, operation 504, operation 404, or any combination thereof includes processing sensor data by one or more machine learning models at operation 1302. After operation 1302, the process 1300 moves to predict a risk of dry-eye based on the processed data in operation 1304. If a risk of dry-eye has been determined in operation 1304, process 1300 goes forward to operation 1306, where the display 310 brightness is flashed to the user of the HMD 302 during operation 1306. In some examples, after flashing the display brightness at operation 1306, the process 1300 can revert back to operation 1302 where the process is repeated.

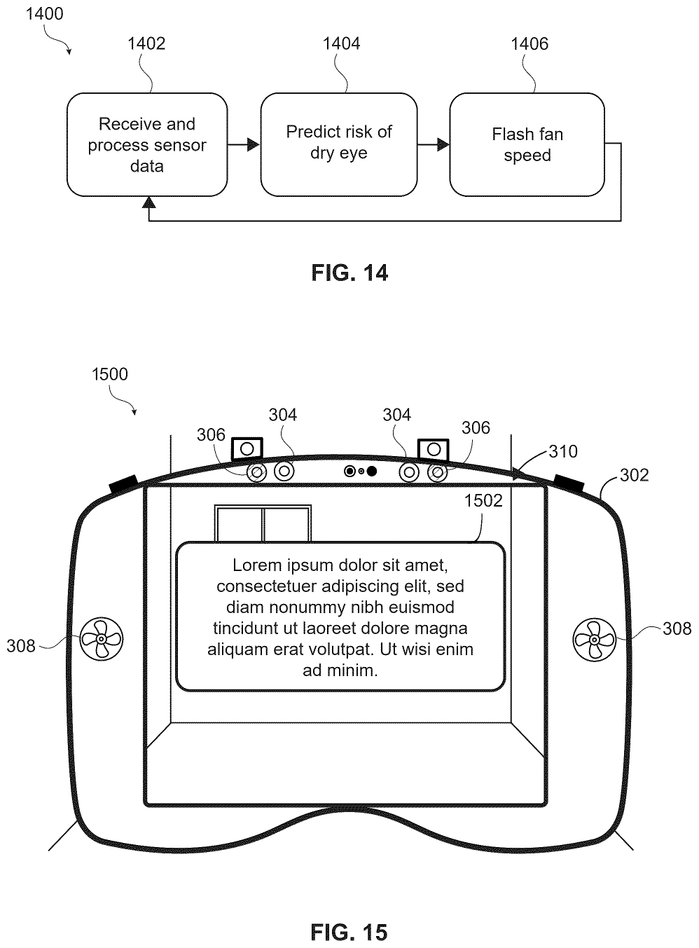

FIG. 14 illustrates an example process 1400 for adjusting the speed of the fan to mitigate a dry-eye condition according to one or more examples of the disclosure. In one or more examples, process 1400 of FIG. 14 receives sensor data and processes the sensor data using one or more machine learning models (e.g., implemented as hardware or using hardware to implement software and/or firmware) at operation 1402 to predict a risk of dry-eye at operation 1404. Process 1400 then moves to operation 1406, where the operation 1406 adjusts the speeds of fans 308 of the HMD 302 according to one or more examples of the disclosure. In one or more examples, once one or more criteria for predicting risk of dry-eye over a threshold of period of time (e.g., 1 second, 1 minute, 1 hour, etc.) is satisfied, the speeds one or more fans 308 of the HMD 302 may be increased to direct more airflow at operation 1406 toward the one or both eyes of the user. Directing increased airflow at operation 1406 to the eyes may modify blinking behavior of the user, including inducing the user to increase blink frequency, thereby mitigating or reducing dry-eye levels as tear production may increase due to the increased blinking frequency. In one or more examples, fan 308 speeds may be lowered to prevent increased dry-eye levels. For example, if increasing the fan 308 speeds at operation 1406 is not increasing blink frequency, the fan speeds may be lowered below a predetermined threshold value to prevent increased dry-eye levels by lowering the cooling rate due to reduced evaporation of tears caused by the reduced airflow. In one or more examples, operation 1406 may adjust the speeds of the one or more fans 308 independently of each other based on the dry-eye levels of each eye. For example, if a left eye is experiencing a higher dry-eye level compared to a right eye, the speed of fan 308 directed toward the left eye may be adjusted while the speed of fan 308 directed toward the right eye may not.

FIG. 15 illustrates an example representation 1500 illustrating a visual notification 1502 displayed by the electronic device to instruct the user on a mitigation for improving a dry-eye condition according to examples of the disclosure. In one or more examples, in contrast to the example of FIG. 11, rather than provide the user with a guided exercise to prevent dry eye that the user can perform while wearing the electronic device, the visual notification 1502 can include a warning to the user, instructing to limit their use of the electronic device. In one or more examples, the electronic device can display notification 1502 in response to predicting a risk of a dry-eye condition. For instance, as an example, the electronic device may display messages such as “Remember to take a break occasionally while wearing the HMD to rest your eyes. If you are feeling symptoms of dry-eye, you can apply a warm compress to your eyes, use tear drops, or rest your eyes.” In one or more examples, the visual notification 1502 is accompanied by a notification sound, haptic feedback, or any combination thereof that is configured to emphasize the visual notification 1502. In one or more examples, the visual notification 1502 is displayed on the display 310 of the HMD 302 periodically as dry-eye levels are continuously monitored. In one or more examples, the visual notification 1502 includes a selectable button which gives the user an option to close or mute the notification 1502. In one or more examples, the electronic device in addition to providing visual notification 1502 may prevent further use of the HMD 302 without determining a reduction in current dry-eye levels. In one or more examples, the visual notification 1502 may provide relevant info such as the user's eye metrics described above (e.g., cooling rate), as well as provide historical data on the user's previous determined dry-eye incidents.

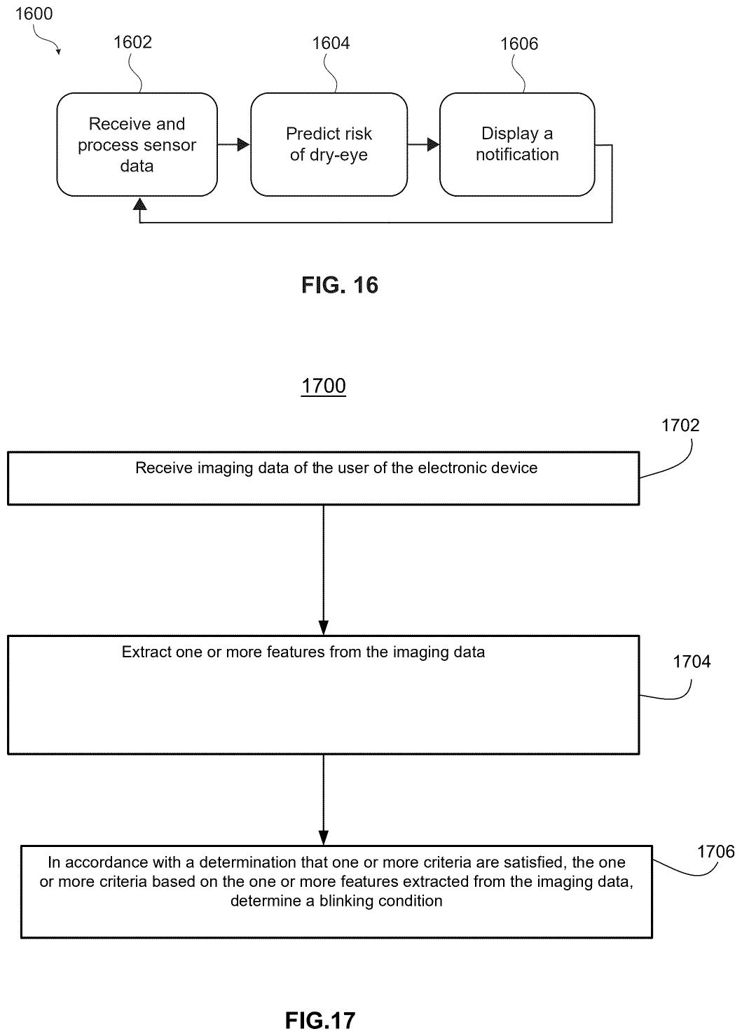

In one or more examples, and as illustrated by the example process 1600, FIG. 16, after operation 1602 receives sensor data and processes the sensor data using one or more machine learning models (e.g., implemented as hardware or using hardware to implement software and/or firmware) at operation 1602, a risk of dry-eye is predicted at operation 1604, the notification 1502 is displayed on the display 310 at operation 1606, and dry-eye levels are checked after a certain period of time (e.g., 1 minute, 5 minutes, etc.), and if dry-eye levels are still 1502 a threshold, the notification 1502 is continued to be displayed. If the notification 1502 was closed after the previous instance, the notification 1502 may reappear if it has not been muted by the user. In one or more examples, additionally or alternatively to displaying a notification, the electronic device can modify blinking behavior of the user, including inducing the user to blink by adjusting and/or modulating the speed of one or more fans of the device that provide air flow to eyes of the user. In one or more examples, extracting features, or receiving and processing sensor data at operation 404, operation 504, operation 1004, operation 1006, operation 1302, operation 1402, operation 1602, or any combination thereof includes processing by one or more machine learning models at operation 1402. In one or more examples, determining a blinking condition and predicting a risk of dry-eye at operation 406, operation 508, operation 1008, operation 1304, operation 1404, operation 1604, or any combination thereof further comprises providing one or more mitigations at operation 1306, operation 1406, operation 1606, or any combination thereof, including displaying notification 1102.

FIG. 17 illustrates an example method for determining a blinking condition according to examples of the disclosure. In one or more examples, the process 1700 is performed at an electronic device in communication with one or more displays and/or one or more input devices including one or more image sensors configured to capture imaging data of one or both eyes of a user of the electronic device. For example, the electronic device is a mobile device (e.g., a tablet, a smartphone, a media player, or a wearable device) including wireless communication circuitry, optionally in communication with one or more of a mouse (e.g., external), trackpad (optionally integrated or external), touchpad (optionally integrated or external), remote control device (e.g., external), another mobile device (e.g., separate from the electronic device), a handheld device (e.g., external), and/or a controller (e.g., external), etc. In one or more examples, the display generation component is a display integrated with the electronic device (optionally a touch screen display), external display such as a monitor, projector, television, or a hardware component (optionally integrated or external) for projecting a user interface or causing a user interface to be visible to one or more users, etc. In one or more examples, the electronic device is part of an electronic device that is part of a wearable device. Examples of input devices include an image sensor (e.g., a camera), thermal sensor, spectrophotometer, location sensor, hand tracking sensor, eye-tracking sensor, motion sensor (e.g., hand motion sensor) orientation sensor, microphone (and/or other audio sensors), touch screen (optionally integrated or external), remote control device (e.g., external), another mobile device (e.g., separate from the electronic device), a handheld device (e.g., external), and/or a controller.

In one or more examples, the electronic device receives, at 1702, the imaging data of the user of the electronic device, including thermal imaging data. In some examples, the one or more image sensors can include Indium Gallium Arsenide (“InGaAs”) photodetectors or any other type of imaging sensors, such as a passive or an active infrared (IR) sensor, for detecting IR light (e.g., visible light sensor, IR sensor, etc.) including infrared ocular thermal imaging sensors. For example, an active IR sensor includes an IR emitter for emitting infrared light into the real-world environment. In one or more examples, the image sensors can include a visible light camera that detects visible light reflecting off the eyes of the user. The image sensors may be placed such that the sensors have a clear and unobstructed view of one or both eyes of the user thus allowing the sensors to be used to take measurements of the one or both eyes during operation of the electronic device. The image sensors may record video and take photos as directed by the device (e.g., the electronic device is communicatively coupled to the thermal image sensor and is configured to send commands to the image sensor to take photos and/or video). In some examples, the thermal image sensors may record data on the infrared energy, or heat signature, of the one or both eyes of the user of the electronic device, including an electronic image. In some examples, the image sensors optionally include one or more visible light image sensors, such as charged coupled device (CCD) sensors, and/or complementary metal-oxide-semiconductor (CMOS) sensors operable to obtain images of the eye(s) of the user of the electronic device. Image sensors also optionally include one or more cameras configured to capture movement of physical objects in the real-world environment. Image sensors also optionally include one or more depth sensors configured to detect the distance of physical objects from electronic device. In some examples, information from one or more depth sensors can allow the device to identify and differentiate objects in the real-world environment from other objects in the real-world environment. In some examples, one or more depth sensors can allow the device to determine the texture and/or topography of objects in the real-world environment.

In one or more examples, the electronic device extracts, at 1704, one or more features from the imaging data. In some examples, the extracted features are stored on the electronic device and/or a storage device in communication with the electronic device. In some examples, the storage device is a cloud storage device including a database of related content. In some examples, the extracted features may include various physical properties of the eye(s) of the user. The physical properties that are extracted can include but are not limited to temperature, moisture, size, shape, color (including ultraviolet, visible, and infrared light), texture, and/or luster. In some examples, the one or more features include patterns extracted from the imaging data, including threshold values.

In one or more examples, in accordance with a determination that one or more criteria are satisfied, the one or more criteria based on the one or more features extracted from the imaging data, the electronic device determines, at 1706, a blinking condition that may predict a risk of dry-eye. Additionally, in one or more examples, in accordance with a determination that one or more criteria are satisfied, the electronic device provides one or more mitigations. In one or more examples, in accordance with a determination that one or more criteria are not satisfied, the electronic device forgoes predicting a risk of a dry-eye condition.

In one or more examples, a dry-eye condition, also referred to as dry-eye syndrome, dry-eye disease, or keratoconjunctivitis sicca (KCS), is an ocular disorder characterized by an imbalance in the tear film of one or both eyes, leading to inadequate lubrication, hydration, and moisture of the eyes. This imbalance may be caused by a variety of factors including but not limited to: decreased tear production, altered tear composition, and increased evaporation of tears. In one or more examples, the tear film is a mixture of water, mucins, lipids, and electrolytes that provides functions such as lubrication nourishment, and protection to the eyes. In one or more examples, in a healthy eye (e.g., pre-determined thresholds based on empirical study or based on baselines for the user of the electronic device), the tear film is renewed through a process called blinking, which helps to maintain optimal (e.g., pre-determined threshold based on empirical study or based on a baseline for the user of the electronic device) moisture levels. In one or more examples, in individuals (or users) with a dry-eye condition, this balance may be disrupted, leading to a range of symptoms including but not limited to: dryness and irritation, burning or stinging sensations, redness and inflammation, blurred vision or sensitivity to light, difficulty wearing contact lenses, and increased risk of corneal ulcers. In one or more examples, and as described above, an underlying cause of a dry-eye condition may include a tear film imbalance. In one or more examples, tear film imbalance may be caused by an increase in tear evaporation due to factors such as environmental dryness, airflow (e.g., wind), exposure to irritants, or lack of sufficient (e.g., pre-determined threshold based on empirical study or based on a baseline for the user of the electronic device) blinking. As described in detailed below, there are various mitigations available for managing or preventing a dry-eye condition, including but not limited to: modifying blinking behavior of the user, including inducing blinking, guided blinking practices, and more, according to some examples of the disclosure.

In some examples, the electronic device is a mobile device (e.g., a head-mounted device, a smart glass, a tablet, a smartphone, a media player, or a wearable device) including wireless communication circuitry, optionally in communication with one or more of a mouse (e.g., external), trackpad (optionally integrated or external), touchpad (optionally integrated or external), remote control device (e.g., external), another mobile device (e.g., separate from the electronic device), a handheld device (e.g., external), and/or a controller (e.g., external), etc., In one or more examples, the display generation component is a display integrated with the electronic device (optionally a touch screen display), external display such as a monitor, projector, television, or a hardware component (optionally integrated or external) for projecting a user interface or causing a user interface to be visible to one or more users, etc. In one or more examples, the electronic device is part of an electronic device that is part of a wearable device. Examples of input devices include an image sensor (e.g., a camera), thermal sensor, spectrophotometer, location sensor, hand tracking sensor, eye-tracking sensor, motion sensor (e.g., hand motion sensor) orientation sensor, microphone (and/or other audio sensors), touch screen (optionally integrated or external), remote control device (e.g., external), another mobile device (e.g., separate from the electronic device), a handheld device (e.g., external), and/or a controller.

Image sensors(s) optionally include one or more visible light image sensors, such as charged coupled device (CCD) sensors, and/or complementary metal-oxide-semiconductor (CMOS) sensors operable to obtain images of the eye(s) of the user of the electronic device. Image sensor(s) also optionally include one or more cameras configured to capture movement of physical objects in the real-world environment. Image sensor(s) also optionally include one or more depth sensors configured to detect the distance of physical objects from electronic device. In some examples, information from one or more depth sensors can allow the device to identify and differentiate objects in the real-world environment from other objects in the real-world environment. In some examples, one or more depth sensors can allow the device to determine the texture and/or topography of objects in the real-world environment. In some examples, the image sensor(s) optionally includes thermal image sensor(s). In some examples, the thermal image sensor(s) can include Indium gallium arsenide (herein referred to as InGaAs) or any other type of thermal imaging sensor(s), such as a passive or an active IR sensor, for detecting infrared light (e.g., visible light sensor, infrared sensor, etc.) including infrared ocular thermal imaging sensor(s). For example, an active IR sensor includes an IR emitter for emitting infrared light into the real-world environment. The thermal image sensor(s) may be placed such that the sensor(s) have a clear and unobstructed view of one or both eyes of the user thus allowing the sensor(s) to be used to take measurements of the eye during operation of the electronic device. The thermal image sensor(s) may record video and take photos as directed by the device (e.g., the electronic device is communicatively coupled to the thermal image sensor and is configured to send commands to the image sensor to take photos and/or video). In some examples, the thermal image sensor(s) may record data on the infrared energy, or heat signature, of the one or both eyes of the user of the electronic device, including an electronic image. In some examples, the data collected from the thermal image sensor(s) may be combined with the data collected from the visible image sensor(s) to produce electronic images blending the temperature and image data. In some examples, the blended temperature and image data includes a color map of the apparent temperature of the eye(s) of the user of the electronic device.

In some examples, the extracted features are stored on the electronic device and/or a storage device in communication with the electronic device. In some examples, the storage device is a cloud storage device including a database of related content. In some examples, the extracted features may include various physical properties of the eye(s) of the user. The physical properties that are extracted can include but are not limited to temperature, moisture, size, shape, color (including ultraviolet, visible, and infrared light), texture, and/or luster. In some examples, the one or more features include patterns extracted from the thermal imaging data, including threshold values.

In some examples, deep learning and/or machine learning method(s) (e.g., a machine learning model implemented as hardware or using hardware to implement software and/or firmware) are used to determine a blinking condition based on the one or more extracted features. For example, the electronic device can include one or more machine learning algorithms such as neural networks (e.g., a convolutional neural network model implemented as hardware or using hardware to implement software and/or firmware), supervised and/or unsupervised machine learning algorithms, and/or the like. The machine learning method(s) may use numerical, categorical, time-series, and/or text data. In one or more examples, the machine learning model(s) may be trained on the cloud, connected to the cloud during use, trained locally, and/or a combination thereof. In one or more examples, threshold factors of the extracted features are determined for determining a blinking condition. For example, threshold factors can include but are not limited to blink frequency, blink completion, spatial properties of tear film temperature regions (e.g., shape, size, etc.), cooling rate, temperatures at various regions of interest, eye redness, blood vessel dilation, etc. The threshold values of the determined threshold factors can be predetermined based on the specific attributes of the user of the electronic device, including but not limited to: age, sex, race, ethnicity, geography, user history, and other information.

In some examples, this method may be used in determining the blinking condition and predicting the risk of dry-eye, as the method allows for assessment of how the eyes of the user of the electronic device open and close. In some examples, the classification process involves identifying multiple states of the first eye over a specific period of time (e.g., a minute, hour, etc.), including both an open state and a closed state. In some examples, these states may be used in determining the blinking condition and predicting the risk of dry-eye or other eye conditions, as they enable the method to distinguish between instances where the user's eyes are open or closed for extended periods, which can be indicative of increased dryness. In some examples, the plurality of states includes partial open states to provide understanding of eye behavior. Partial open states may further be specified to partial open right and partial open left, with each state specifying which eye is partially open and which is fully open. In some examples, the method includes ensuring that the image sensors are capable of capturing imaging data that allows for feature extraction and assessment. In some examples, the method may utilize machine learning or deep learning techniques (e.g., a convolutional neural network model implemented as hardware or using hardware to implement software and/or firmware) to improve the confidence of the assessment and predict the risk of dry-eye, including handling variations in lighting conditions, eye movement, and other environmental factors that may impact the confidence of assessment.

In some examples, combining the plurality of states may allow for an understanding of overall eye behavior, which may be useful in predicting the risk of dry-eye and determining the likelihood of blinking. In some examples, the combined representation may be calculated by averaging or aggregating the classification states for the two eyes over a specific period of time (e.g., a minute, hour, etc.). In some examples, this provides a snapshot of the user's overall eye behavior which can be used to inform decisions related to dry-eye mitigation or other applications. In some examples, the filtering step allows for the removal of noise or irrelevant data that may impact the accuracy of the analysis. In some examples, the filtering step may remove instances where the eyes are not fully open or closed, or where there is significant eye movement or other environmental factors that may affect the classification results.

In some examples, peak detection may allow for the identification of specific patterns or events in the user's eye behavior that may be indicative of certain conditions, such as a blink or a dry-eye condition. In some examples, the peak detection step involves identifying local maxima or minima in the combined representation after filtering. In some examples, these peaks can correspond to instances where the eyes are fully open or closed, or where there is sudden change in eye movement or other environmental factors that may impact the classification results. In some examples, a peak in the combined representation may indicate a moment of rapid eye movement or a brief period of eye closure, which could be indicative of a blink condition. In some examples, detection of peaks can be performed using various techniques. In some examples, these techniques include but are not limited to local maximum/minimum detection and peak-finding algorithms. In some examples, local maximum/minimum detection may involve identifying local maxima or minima in the combined representation by comparing adjacent data points. Peak-finding algorithms can identify patterns or events in the combined representation that may correspond to peaks. The advantages of this method can include ability to detect specific patterns or events in user eye behavior.

In some examples, a secondary classification step may allow for the identification of complete and incomplete blink events, which can be used to inform decisions related to dry-eye risk or other applications. In some examples, a secondary classification step involves classifying the detected peaks as either complete or incomplete blink events for each eye. In some examples, a complete blink event is defined as a peak that corresponds to a full closure of both eyes, while an incomplete blink event may be a peak that corresponds to only one eye being fully closed. In some examples, a peak may be classified as an incomplete blink event for the first eye if the peak corresponds to the first eye being closed and second eye remaining open or partially closed. In some examples, classification of peaks as complete or incomplete blink events can be performed using various techniques, including but not limited to machine learning (e.g., a convolutional neural network model implemented as hardware or using hardware to implement software and/or firmware) algorithms and rule-based systems. In some examples, machine learning algorithms may be trained on a dataset of labeled examples to learn patterns in the combined representation that distinguish between complete and incomplete blink events. By classifying peaks as complete or incomplete blink events, the method may provide insights into overall eye function, enabling targeted interventions to maintain healthy (e.g., pre-determined threshold based on empirical study or based on a baseline for the user of the electronic device) eye function.

In some examples, the extracting of spatial properties refers to a wide range of characteristics that may describe the shape, size, position, orientation, and relationships between various features of the eyes and surrounding tissues. In some examples, the extraction of spatial properties of the eyes involves analyzing the imaging data captured by the image sensors to identify features such as: eye size and shape, including vertical and horizontal dimensions, elliptical or spherical shape; eye position and orientation, including rotation, tilt, and translation relative to the face and surroundings; distance between the eyes; shape and size of the eyelids, including upper and lower eyelids, lid margins, and orbital rim; shape and size of the pupils, including diameter and shape; iris texture and pattern, including freckles, striations, and color; corneal curvature and shape, including radius of curvature, vertex distance, and astigmatism; conjunctival folds and creases; orbital rim and facial structure, including bone shape and size, muscle attachments; eye movement patterns and ranges of motion, including horizontal and vertical gaze, saccadic movements; blinking patterns and frequencies, including complete blink events and incomplete blink events for each eye, frequency, duration, and amplitude or strength of blinks, relationships between blinking and other eye movements. In some examples, the method can also extract spatial properties related to the cornea, providing an understanding of the corneal shape, surface topography, and reflective characteristics. In some examples, extracted features include but are not limited to corneal curvature and shape, including radius of curvature, vertex distance, and astigmatism; corneal surface topography, including ridges, valleys, and elevations that can indicate irregularities or abnormalities (e.g., pre-determined threshold based on empirical study or based on a baseline for the user of the electronic device) in the epithelial layer or stroma; and corneal glints, which are reflections of light off the corneal surface, including intensity, duration, and pattern of reflection. In some examples, the method can identify anterior corneal glints, such as tear film reflections or epithelial cell layer reflections, which can indicate the presence of dry-eye or other corneal disorders, as well as posterior corneal glints, such as various membrane reflections, which can provide insight into the structure and function of the corneal layers. In some examples, by extracting these spatial properties, the method can provide an understanding of the cornea and its relationship to overall eye function, enabling targeted interventions to maintain healthy eye function and diagnose potential corneal disorders. In some examples, the method involves extracting one or more features from thermal imaging data, focusing specifically on the spatial properties of tear film temperature regions of the eyes of the user of the electronic device. The thermal imaging data may be captured using thermal sensors or cameras that detect variations in temperature across the surface of the eyes. The data is then processed to identify distinct regions of the tear film that exhibit varying temperature characteristics. In some examples, the spatial properties refer to geometric and positional attributes, such as the shape, size, location, and distribution patterns of the tear film temperature regions within the thermal image. These properties may be quantified through image processing techniques that segment and analyze the tear film based on identified temperature variations. For example, one or more regions of the eye may be segmented based on the temperature within a threshold margin of variance in temperature. In one or more examples, a cooling rate is determined for each temperature region. In some examples, the analysis of spatial properties of tear film temperature regions is utilized for diagnostic or monitoring purposes. In some examples, the extracted spatial properties of the tear film temperature regions are correlated with specific physiological conditions of the eyes. The method may utilize these spatial properties to assess tear film stability, hydration, or other relevant ocular metrics. In some examples, image processing techniques, including thermal gradient analysis, segmentation algorithms, and pattern recognition methods, are employed to extract spatial properties from the thermal imaging data. In some examples, processing steps may include filtering, image thresholding, edge detection, region growing methods, watershed segmentation, or feature extraction algorithms designed to identify and isolate relevant temperature regions within the tear film. These segmentation techniques may be used alone or in combination with each other or with additional processing steps to identify the one or more tear film temperature regions. In some examples, spatial properties of the identified tear film regions may include, but are not limited to: temperature values, spatial distributions (e.g., heat maps), region sizes and shapes boundaries and contours. In some examples, the method may further comprise analyzing the segmented tear film regions to identify patterns or anomalies that indicate a potential dry-eye condition. This analysis may be performed using various techniques, including but not limited to: statistical processing, machine learning algorithms (e.g., a convolutional neural network model implemented as hardware or using hardware to implement software and/or firmware), and pattern recognition methods. In some examples, results of this analysis can be used to provide feedback to the user, such as alerts or warnings about potential dry-eye conditions, and/or to trigger additional processing steps, such as generating a report or sending data to a healthcare provider. In some examples, the shape of the tear film regions can be analyzed to determine various characteristics, such as boundary irregularities, region convexity and concavity, shape asymmetry, size, and aspect ratio. In some examples, these shape-based properties can be used to identify potential dry-eye conditions by comparing them to known shapes associated with normal (e.g., pre-determined threshold based on empirical study or based on a baseline for the user of the electronic device) tear film regions. In some examples, the method may further comprise analyzing the shape-based properties to detect changes in the tear film regions over time, such as changes in boundary and perimeter irregularities, shifts in convexity and concavity, or alterations in shape asymmetry. In some examples, these changes can be indicative of a developing dry-eye condition, and the method may trigger alerts or warnings accordingly. In some examples, these shape-based properties can be used to track the effectiveness of treatment for dry-eye conditions. In some examples, identified shapes can also be used to compare with known and abnormal (e.g., pre-determined threshold based on empirical study or based on a baseline for the user of the electronic device) tear film region shapes in a database, allowing for identification of potential dry-eye conditions. The comparison can be performed using various techniques, including but not limited to: image processing algorithms, machine learning models (e.g., a convolutional neural network model implemented as hardware or using hardware to implement software and/or firmware), neural networks, or statistical analysis methods. The size of the tear film regions can be analyzed to determine various characteristics, such as: size variation between regions, regional growth or shrinkage over time, and relative sizes of adjacent regions. These size-based properties can be used to identify potential eye conditions by comparing them to known sizes associated with normal tear film regions. For example, an abnormally large or small region may indicate a developing eye condition. In some examples, the method may further comprise analyzing the size-based properties to detect changes in the tear film regions over time, such as: changes in regional growth or shrinkage rates, shifts in relative sizes of adjacent regions, or alterations in overall tear film volume. In some examples, these changes can be indicative of a developing eye condition, and the method may trigger alerts or warnings accordingly. The identified size properties can also be used to compare with known normal and abnormal tear film region sizes in a database, allowing for identification of potential eye conditions. The comparison can be performed using various techniques, including but not limited to: image processing algorithms, machine learning models (e.g., implemented as hardware or using hardware to implement software and/or firmware), neural networks, or statistical analysis methods. In some examples, these spatial properties may include characteristics such as tear film area and perimeter, which can be calculated using various techniques, including but not limited to: image processing algorithms, neural networks, or statistical analysis methods. The tear film area and perimeter can provide information about the one or both eyes of the user, particularly in relation to dry-eye conditions. For example, an abnormal or high ratio (e.g., pre-determined threshold based on empirical study or based on a baseline for the user of the electronic device) of tear film area to perimeter may indicate a developing dry-eye condition. The method may compare the ratio to a baseline threshold value, which can be determined by various methods. The threshold value may be adjusted based on various factors specific to the user, including but not limited to age, gender, sex, race, ethnicity, location, user history, and environmental conditions. In some examples, the threshold value is determined using statistical analysis, machine learning algorithms, image processing techniques, clinical trials, and more. In some examples, the ratio of the area to the perimeter of the extracted tear film region is used to determine dry-eye levels. This determination can include determining even or uneven cooling of the eye. In one or more examples, a high tear film ratio can indicate dry-eye, whereas a lower tear film ratio can act as a normal baseline threshold (e.g., pre-determined threshold based on empirical study or based on a baseline for the user of the electronic device) value depending on the user of the electronic device.

In some examples, thermal data extracted by a thermal sensor may be used to detect blinks by measuring the heat generated by the rubbing of one or more eyelids. In some examples, a heat map of the eye is generated to detect blink completeness. In some examples, the image data may be combined with the thermal data to provide higher confidence in determining a blinking condition. In some examples, the visible light sensor may operate independently of the thermal image sensor. In some examples, the imaging data and thermal data may be processed differently. In some examples, the method involves extracting one or more features from thermal imaging data, focusing specifically on the spatial properties of tear film temperature regions of the eyes of the user of the electronic device. The thermal imaging data may be captured using thermal sensors or cameras that detect variations in temperature across the surface of the eyes. The data is then processed to identify distinct regions of the tear film that exhibit varying temperature characteristics. In some examples, the spatial properties refer to geometric and positional attributes, such as the shape, size, location, and distribution patterns of the tear film temperature regions within the thermal image. These properties may be quantified through image processing techniques that segment and analyze the tear film based on identified temperature variations. In some examples, the analysis of spatial properties of tear film temperature regions is utilized for diagnostic or monitoring purposes. In some examples, the extracted spatial properties of the tear film temperature regions are correlated with specific physiological conditions of the eyes. The method may utilize these spatial properties to assess tear film stability, hydration, or other relevant ocular metrics. In some examples, image processing techniques, including thermal gradient analysis, segmentation algorithms, and pattern recognition methods, are employed to extract spatial properties from the thermal imaging data. The processing steps may include filtering, image thresholding, edge detection, region growing methods, watershed segmentation, or feature extraction algorithms designed to identify and isolate relevant temperature regions within the tear film. These segmentation techniques may be used alone or in combination with each other or with additional processing steps to identify the one or more tear film temperature regions. The spatial properties of the identified tear film regions may include, but are not limited to: temperature values, spatial distributions (e.g., heat maps), region sizes and shapes boundaries and contours. In some examples, the method may further comprise analyzing the segmented tear film regions to identify patterns or anomalies that indicate a potential dry-eye condition. This analysis may be performed using various techniques, including but not limited to: statistical processing, machine learning algorithms, and pattern recognition methods. The results of this analysis can be used to provide feedback to the user, such as alerts or warnings about potential dry-eye conditions, and/or to trigger additional processing steps, such as generating a report or sending data to a healthcare provider. The shape of the tear film regions can be analyzed to determine various characteristics, such as boundary irregularities, region convexity and concavity, shape asymmetry, size, and aspect ratio. These shape-based properties can be used to identify potential dry-eye conditions by comparing them to known shapes associated with normal tear film regions. In some examples, the method may further comprise analyzing the shape-based properties to detect changes in the tear film regions over time, such as changes in boundary and perimeter irregularities, shifts in convexity and concavity, or alterations in shape asymmetry. Additionally, these shape-based properties can be used to track the effectiveness of treatment for dry-eye conditions. The identified shapes can also be used to compare with known and abnormal tear film region shapes in a database, allowing for identification of potential dry-eye conditions. The comparison can be performed using various techniques, including but not limited to: image processing algorithms, machine learning models (e.g., implemented as hardware or using hardware to implement software and/or firmware), neural networks, or statistical analysis methods. The size of the tear film regions can be analyzed to determine various characteristics, such as: size variation between regions, regional growth or shrinkage over time, and relative sizes of adjacent regions. These size-based properties can be used to identify potential eye conditions by comparing them to known sizes associated with normal tear film regions. For example, an abnormally large or small region may indicate a developing eye condition. In some examples, the method may further comprise analyzing the size-based properties to detect changes in the tear film regions over time, such as: changes in regional growth or shrinkage rates, shifts in relative sizes of adjacent regions, or alterations in overall tear film volume. These changes can be indicative of a developing eye condition, and the method may trigger alerts or warnings accordingly. The identified size properties can also be used to compare with known normal and abnormal tear film region sizes in a database, allowing for identification of potential eye conditions. The comparison can be performed using various techniques, including but not limited to: image processing algorithms, machine learning models (e.g., implemented as hardware or using hardware to implement software and/or firmware), neural networks, or statistical analysis methods. In some examples, these spatial properties may include characteristics such as tear film area and perimeter, which can be calculated using various techniques, including but not limited to: image processing algorithms, neural networks, or statistical analysis methods. The tear film area and perimeter can provide information about the one or both eyes of the user, particularly in relation to dry-eye conditions. For example, an abnormal or high ratio of tear film area to perimeter may indicate a developing dry-eye condition. The method may compare the ratio to a baseline threshold value, which can be determined by various methods. The threshold value may be adjusted based on various factors specific to the user, including but not limited to age, gender, sex, race, ethnicity, location, user history, and environmental conditions. In some examples, the threshold value is determined using statistical analysis, machine learning algorithms, image processing techniques, clinical trials, and more. In some examples, the ratio of the area to the perimeter of the extracted tear film region is used to determine dry-eye levels. This determination can include determining even or uneven cooling of the eye. In one or more examples, a high tear film ratio can indicate dry-eye, whereas a lower tear film ratio can act as a normal baseline threshold value depending on the user of the electronic device.

In some examples, blinking frequency may refer to the number of blinks per unit time, which can be measured in blinks per minute (bpm) or blinks per second (bps). This information can indicate normal or abnormal (e.g., pre-determined threshold based on empirical study or based on a baseline for the user of the electronic device) blinking patterns, such as an increased or decreased blink rate. For example, a high blinking frequency may indicate anxiety or stress, while a low blinking frequency may indicate dry-eye or other ocular disorders. In some examples, blinking duration refers to the length of time taken for the eyelids to close and reopen during a blink cycle. This information can provide insight into the completeness of blinks, as well as any irregularities in the blink pattern. For example, a short blinking duration may indicate incomplete blinks or rapid eye movements, while a long blinking duration may indicate prolonged eye closure. In some examples, blinking completeness refers to the extent to which the eyelids close and reopen during a blink cycle. In some examples, blinking completeness may also factor partially or fully complete blinks for either eye, and the combinations thereof. This information can provide insight into the overall health of the eyes and surrounding tissues, as well as any potential issues related to dry-eye or other ocular disorders. For example, incomplete blinks may indicate corneal surface irregularities or tear film abnormalities, while complete blinks may indicate normal (e.g., pre-determined threshold based on empirical study or based on a baseline for the user of the electronic device) eye function. In some examples, the method can use blinking frequency, duration, and completeness in combination with other spatial properties and thermal imaging data to analyze eye function and behavior. For example, a high blinking frequency combined with short blinking durations and incomplete blinks may indicate anxiety or stress, while a low blinking frequency combined with long blinking durations and complete blinks may indicate dry-eye or other ocular disorders. By analyzing these features in combination, the method can provide an understanding of eye function and behavior, enabling targeted interventions to maintain healthy eye function. The extracted features can be used in conjunction with other claims related to image processing, machine learning, and blink detection to develop more models of eye function and predict potential issues related to dry-eye or other conditions. In some examples, the method extracts thermal properties from the imaging data, including temperature, heat flux, and thermal conductivity, and change in temperature/thermal properties of one or both eyes of the user of the electronic device. This information can provide insight into the physiological state of the eyes and surrounding tissues, such as corneal surface temperature and thermal conductivity, tear film thickness and refractive index, conjunctival and corneal blood flow and oxygen saturation, and orbital rim and facial structure temperature and thermal conductivity. The method can also extract blinking frequency, duration, and completeness of the one or both eyes of the user, in addition to other spatial properties such as eye size and shape, position and orientation, distance between the eyes, shape and size of the eyelids, shape and size of the pupils, iris texture and pattern, corneal curvature and shape, conjunctival folds and creases, orbital rim and facial structure. By extracting these thermal properties and thermal imaging data, the method can provide an understanding of eye function and behavior, enabling targeted interventions to maintain healthy eye function. In some examples, the extracted features can be used in conjunction with other claims related to image processing, machine learning, and blink detection to develop more models of eye function and predict potential issues related to dry-eye or other conditions. In some examples, the method can use thermal imaging data to analyze changes in temperature and heat flux over time, allowing for the detection of anomalies or abnormalities (e.g., pre-determined threshold based on empirical study or based on a baseline for the user of the electronic device) in eye function. In some examples, each user may have a specific baseline of overall blinking frequency, duration, and/or completeness. Deviating from the baseline specific to the user may be an indication of an eye condition. For example, a detection of blinking frequency higher than the users baseline blinking frequency may indicate a dry-eye condition. This information can be used to inform decisions related to dry-eye risk and mitigation or other applications, such as recommending specific treatments or therapies based on the user's eye health.

In some examples, blinking duration refers to the length of time taken for the eyelids to close and reopen during a blink cycle. In some examples, the blinking duration is a measurement of time (e.g., nanosecond, millisecond, second, etc.). In some examples, the blinking duration is a measurement relative to the device (e.g., frame). This information can provide insight into the completeness of blinks, as well as any irregularities in the blink pattern. In some examples, the method can use blinking duration as an additional criterion in combination with other spatial properties and thermal imaging data, such as blink strength, eye size and shape, position and orientation, distance between the eyes, shape and size of the eyelids, shape and size of the pupils, iris texture and pattern, corneal curvature and shape, conjunctival folds and creases, orbital rim and facial structure. For example, in some examples, a short blinking duration may indicate incomplete blinks or rapid eye movements, while a long blinking duration may indicate prolonged eye closure. In some examples, the method can use blinking duration to analyze changes in blink patterns over time, allowing for the detection of anomalies or abnormalities in eye function. In some examples, this information can be used to inform decisions related to dry-eye risk or mitigation or other applications, such as recommending specific treatments or therapies based on the user's eve health, or any combination thereof. In some examples, by using blinking duration as an additional criterion, the method can provide understanding of eye function and behavior, enabling targeted interventions to maintain healthy eye function. The extracted features can be used in conjunction with other methods related to image processing, machine learning, and blink detection to develop more models of eye function and predict potential issues related to dry-eye or other conditions.

In some examples, blinking completeness refers to the extent to which the eyelids close and reopen during a blink cycle. In some examples, blinking completeness may also factor partially or fully complete blinks for either eye, and the combinations thereof. In some examples, the method extracts blinking completeness information from the imaging data and uses blinking completeness information as an additional criterion in the determination of whether the one or both eyes are at risk of dry-eye. The method can use blinking completeness to analyze changes in blink patterns over time, allowing for the detection of anomalies or abnormalities in eye function. The method can also use other spatial properties and thermal imaging data, such as eye size and shape, position and orientation, distance between the eyes, shape and size of the eyelids, shape and size of the pupils, iris texture and pattern, corneal curvature and shape, conjunctival folds and creases, orbital rim and facial structure. For example, a high blinking completeness may indicate normal eye function, while a low blinking completeness may indicate incomplete blinks or rapid eye movements. In some examples, blink completeness is determined based on the presence of the one or more corneal glints of the eyes of the user. For example. A presence of the corneal glint of an eye is detected, that eye may be considered completely or partially open. Not detecting a corneal glint may be an indication of a blink condition. In some examples, blink completeness is determined based on the presence of the one or more iris of the eyes of the user. For example. A presence of the iris of an eye is detected, that eye may be considered completely or partially open. Not detecting an iris may be an indication of a blink condition. By using blinking completeness as an additional criterion, the method can provide an understanding of eye function and behavior, enabling targeted interventions to maintain healthy eye function. The extracted features can be used in conjunction with other claims related to image processing, machine learning, and blink detection to develop more models of eye function and predict potential issues related to dry-eye or other conditions.

In some examples, blinking frequency can be used to inform decisions related to dry-eye mitigation or other applications such as determining a blinking condition. In some examples, the method extracts blinking frequency information from the imaging data and uses the information as an additional criterion in the determination of whether the one or both eyes are at risk of dry-eye. In some examples, blinking frequency refers to the number of blinks per unit time, which can be quantified using various metrics, including but not limited to: blinks per minute, blinks per second, blinking frequency ratio defined as the ratio of blinking frequency to non-blinking frequency, blinking duty cycle defined as the percentage of time spent blinking during a given period, blinking coefficient of variation defined as the standard deviation of blinking frequency divided by its mean, or any combination thereof. Different blink types may be factored into blink frequency, such as incomplete or complete blinks. In some examples, blink frequency may be the number of blinks within a range of time periods (e.g., minute, hour, day, etc.). In some examples, complete and incomplete blinks may be factored into the blink frequency with equal or different coefficients. For example, incomplete blinks may have less weight than complete blinks. In some examples, blink frequency may only include the blinks of a single eye. In some examples, blink frequency may include the average, or a different combination of the blinks of both eyes. In some examples, each eye may have a different blink frequency that may be calculated differently (e.g., different weights for blinks). In some examples, the method can use these various metrics in combination with other spatial properties and thermal imaging data, such as eye size and shape, position and orientation, distance between the eyes, shape and size of the eyelids, shape and size of the pupils, iris texture and pattern, corneal curvature and shape, conjunctival folds and creases, orbital rim and facial structure, and more. For example, in some examples, a high blinking frequency combined with a low blinking duty cycle may indicate dry-eye or other ocular disorders. In some examples, a low blinking frequency combined with a high blinking coefficient of variation may indicate normal eye function. In some examples, by using blinking frequency as an additional criterion, the method can provide an understanding of eye function and behavior, enabling targeted interventions to maintain healthy eye function. The extracted features can be used in conjunction with other claims related to image processing, machine learning, and blink detection to develop more models of eye function and predict potential issues related to dry-eye or other conditions. In some examples, the method can also use blinking frequency as a criterion for determining whether the one or both eyes are in a state of stress or anxiety, based on changes in blinking patterns over time. For example, an increase in blinking frequency may indicate increased stress or anxiety levels, while a decrease in blinking frequency may indicate relaxation or decreased stress levels.

In some examples, the method utilizes application of machine learning models (e.g., implemented as hardware or using hardware to implement software and/or firmware) in feature extraction, enabling the method to learn patterns and relationships within the imaging data. In some examples, the method applies one or more machine learning models to the imaging data to extract the one or more features. In some examples, this can include using techniques such as convolutional neural networks (CNNs) to extract spatial features from the imaging data, recurrent neural networks (RNNs) to extract temporal features from the imaging data, autoencoders to learn compressed representations of the imaging data, random forests or support vector machines (SVMs) to classify the extracted features into different categories, gradient boosting machines to predict continuous values based on the extracted features, and neural networks with transfer learning to leverage pre-trained models for feature extraction, or any combination thereof. In some examples, the machine learning models can be trained using labeled datasets of imaging data, where the labels represent the desired output or classification. This allows the method to learn patterns and relationships within the imaging data that are relevant to the task at hand. In some examples, the electronic device can include one or more machine learning algorithms such as neural networks (e.g., convolutional neural networks), supervised or unsupervised machine learning algorithms, long-term-short-term memory network, or any combination thereof. The machine learning method(s) may use numerical, categorical, time-series, and/or text data. In one or more examples, the machine learning model(s) may be trained on the cloud, connected to the cloud during use, trained locally, and/or a combination thereof. In one or more examples, threshold factors of the extracted features are determined for determining a blinking condition. In some examples, threshold factors can include but are not limited to blink frequency, blink completion, spatial properties of tear film temperature regions (e.g., shape, size, etc.), cooling rate, temperatures at various regions of interest, eye redness, blood vessel dilation, etc. The threshold values of the determined threshold factors can be predetermined based on the specific attributes of the user of the electronic device, including but not limited to age, sex, race, ethnicity, geography, user history, and other information. In some examples, the supervised machine learning (ML) model may include a neural network (e.g., deep learning model, logistic regression, linear or non-linear support vector machine, boosted decision tree, convolutional neural network, gated recurrent network, long short-term memory network, etc.). In some examples, artificial intelligence (AI)/ML systems (e.g., implemented as hardware or using hardware to implement software and/or firmware) may utilize models that may be trained (e.g., supervised learning or unsupervised learning) using various training data, including data collected using a user device. Such use of user-collected data may be limited to operations on the user device. For example, the training of the model can be done locally on the user device so no part of the data is sent to another device. In other implementations, the training of the model can be performed using one or more other devices (e.g., server(s)) in addition to the user device but done in a privacy preserving manner, e.g., via multi-party computation as may be done cryptographically by secret sharing data or other means so that the user data is not leaked to the other devices. In some examples, the machine learning model is a support vector machine. In some examples, the extracted features can be used in conjunction with other claims related to image processing, blink detection, and eye tracking to develop more models of eye function and predict potential issues related to dry-eye or other conditions. In some examples, the method can also use transfer learning to leverage pre-trained models for feature extraction, allowing for faster training times and improved performance. In some examples, the method can use ensemble methods to combine the predictions from multiple machine learning models to improve overall accuracy and robustness.

In some examples, machine learning models (e.g., implemented as hardware or using hardware to implement software and/or firmware) are introduced in evaluating the blinking condition, enabling the method to learn patterns and relationships within the extracted features. In some examples, the method applies one or more machine learning models to the one or more features to evaluate the blinking condition. In some examples, the machine learning models can be trained using labeled datasets of features extracted from imaging data, where the labels represent the desired output or classification (e.g., supervised training). In some examples, the predicted outcomes can be used in conjunction with other methods related to image processing, blink detection, and eye tracking to develop more models of eye function and predict potential issues related to dry-eye or other conditions. In some examples, the machine learning models play a role in evaluating the one or more criteria and determining a blinking condition. In some examples, analyzing the extracted features, these models can identify patterns and relationships, enabling them to predict whether a blinking condition is present or not. For example, a machine learning model trained on thermal imaging data may be able to detect changes in corneal temperature or heat flux that are indicative of dry-eye, even before symptoms become apparent. In some examples, a machine learning model analyzing spatial properties such as eyelid position and shape may be able to identify abnormal blinking patterns that indicate increased risk of dry-eye. In some examples, the method can also use transfer learning to leverage pre-trained models for classification or regression tasks, allowing for faster training times and improved performance. In some examples, the method can use ensemble methods to combine the predictions from multiple machine learning models to improve overall accuracy and robustness. The extracted features can include spatial properties such as eye size and shape, position and orientation, distance between the eyes, shape and size of the eyelids, shape and size of the pupils, iris texture and pattern, corneal curvature and shape, conjunctival folds and creases, orbital rim, facial structure, and more, or any combination thereof. In some examples, the method can also extract thermal imaging data, including temperature, heat flux, and thermal conductivity. In some examples, by combining the predictions from multiple models, the method can provide an evaluation of the one or more criteria and determine whether a blinking condition is present or not, allowing for early intervention and prevention of dry-eye conditions.

In some examples, a support vector machine (SVM) is a ML algorithm used for the classification and outlier detection of data points within a feature space. SVM algorithms can find a hyperplane in an N-dimensional space that can separate data points in different classes in a feature space. The hyperplane of a 2-demensional space can be a line separating two classes or categories of vectors or data points. An optimal hyperplane in a 2-dimensional space is a line that maximizes a distance between the closest data points (or vectors) of different classes in the feature space. In some examples, a one-class SVM may be used as part of the ML models. A one-class SVM can use a kernel function to map input data to a higher-dimensional space where the data points are more separable. The one-class SVM can include a common kernel, such as a linear kernel, polynomial kernel 1, radial bases function (RBF) kernel, or a sigmoid kernel. The training process for building the distribution model can involve fitting the one-class SVM model to the normal data points. An SVM algorithm can find an optimal hyperplane in an N-dimensional space that can separate data points in different classes in a feature space. The hyperplane of a 2-demensional space can be a line separating two classes or categories of vectors or data points. In some examples, an optimal hyperplane in a 2-dimensional space is a line that maximizes a distance between the closest data points (or vectors) of different classes in the feature space.

In some examples, the supervised machine learning (ML) model may include a neural network (e.g., deep learning model, logistic regression, linear or non-linear support vector machine, boosted decision tree, decision tree, random forest, recurrent neural network, transformer, convolutional neural network, gated recurrent network, long short-term memory network, etc.). In some examples, artificial intelligence (AI)/ML systems may utilize models (e.g., implemented as hardware or using hardware to implement software and/or firmware) that may be trained (e.g., supervised learning or unsupervised learning) using various training data, including data collected using a user device. Such use of user-collected data may be limited to operations on the user device. For example, the training of the model can be done locally on the user device so no part of the data is sent to another device. In other implementations, the training of the model can be performed using one or more other devices (e.g., server(s)) in addition to the user device but done in a privacy preserving manner, e.g., via multi-party computation as may be done cryptographically by secret sharing data or other means so that the user data is not leaked to the other devices. In some examples, in place of or in addition to SVMs, one or a combination of many AI models (e.g., support vector machines, decision trees, random forests, neural networks, convolutional neural networks, recurrent neural networks, transformers) that can perform supervised machine learning may be used, in addition to AI models not explicitly mentioned herein.

In some examples, the detection of one or more corneal glints of the one or both eyes of the user of the electronic device as a feature can be used to evaluate blinking behavior and detect potential dry-eye conditions. In some examples, the method detects the presence of one or more corneal glints of the one or both eyes of the user. In some examples, corneal glints refer to the reflection of light off the surface of the cornea, which can provide insight into the health and function of the eyes. In some examples, if no corneal glints are detected, the lack of corneal glint detection may indicate that a blink of the one or both eyes of the user of the electronic device is present or imminent, as the absence of glints can be indicative of closed eyelids. In some examples, if corneal glints are partially detected, partial corneal glints detection may suggest that only a partial or incomplete blink of the one or both eyes of the user of the electronic device has occurred. For example, in some examples, detecting only half of the threshold number of corneal glints may indicate the eye is half or partially closed. In some examples, by detecting the presence and intensity of corneal glints, the method can gain insights into blinking behavior and identify potential issues related to dry-eye or other ocular disorders. In some examples, the extracted features can be used in conjunction with other methods related to image processing, blink detection, and eye tracking to develop more models of eye function and predict potential issues related to dry-eye or other conditions.

In some examples, the detection of one or more pupils of the one or both eyes of the user of the electronic device as a feature can be used to evaluate blinking behavior and detect potential dry-eye conditions. In some examples, the method detects the presence of one or more iris of the one or both eyes of the user. In some examples, pupil refers to the opening in the center of the iris that allows light to enter the eye, which can provide insight into the health and function of the eyes. In some examples, if no pupils are detected, the lack of pupil detection may indicate that a blink of the one or both eyes of the user of the electronic device is present or imminent, as the absence of pupils can be indicative of closed eyelids. In some examples, if pupils are partially detected, partially detected pupils may suggest that only a partial or incomplete blink of the one or both eyes of the user of the electronic device has occurred. For example, in some examples, detecting only half of the threshold number of the pupil may indicate the eye is half or partially closed. In some examples, by detecting the presence and intensity of pupils, the method can gain insights into blinking behavior and identify potential issues related to dry-eye or other ocular disorders. In some examples, the extracted features can be used in conjunction with other methods related to image processing, blink detection, and eye tracking to develop more models of eye function and predict potential issues related to dry-eye or other conditions.

In one or more examples, the electronic device, in response to determining a threshold (e.g., 5 dry-eye conditions) of a risk of dry-eye conditions over a threshold period of time (e.g., a minute, an hour, etc.), provides one or more mitigations based on the dry-eye condition. In one or more examples, setting a threshold number of dry-eye predictions before applying mitigations may reduce a possibility of false positive dry-eye predictions, where the system predicts a dry-eye condition when one is not present. In one or more examples, dry-eye predictions are reduced to a threshold number when there is an increased rate of dry-eye determinations above a threshold number of times (e.g., 5 dry-eye determinations) in a threshold period of time (e.g., a minute, an hour, etc.). In one or more examples, reducing dry-eye predictions after exceeding a threshold number may reduce computational strain on the system, and optionally reduce the drain on a batter if one is present in the system. In some examples, a popup notification includes: providing a notification based on the information, sending a message based on the information, displaying the information, controlling a user interface of an application based on the information, controlling a user interface of a health application based on the information, controlling a focus mode based on the information, setting a reminder based on the information, or adding a calendar entry based on the information, or any combination thereof. In some examples, a notification may be displayed after determining a risk of an eye condition based on the one or more extracted features. Eye condition risk may be determined by detecting a deviance from a predetermined threshold of blinks per minute. In some examples, the threshold number of blinks may be between 12 and 25 blinks per minute. In some examples, the threshold may be between 1 and 60 blinks per minute, or any other reasonable threshold. The threshold values may be adjusted dynamically, or buffer values may be added on either the lower or upper limit to avoid false positives. In some examples, a notification may be displayed when blinking is within threshold values. Notifying when blinking is within threshold values may indicate normal (e.g., pre-determined threshold based on empirical study or based on a baseline for the user of the electronic device) blinking pattern. In some examples, the notification may inform the user to apply a warm compress to the eyes. In some examples, warnings, alerts, and notifications may include information about treatments, therapies, or strategies that address the underlying causes of dry-eye, such as artificial tears, ointments, warm compresses, eye massage, humidity therapy, blinking exercises, avoiding irritants, using anti-inflammatory medications, and more. In some examples, warnings and alerts recommend the user to seek a healthcare professional. In some examples, the method may allow the user to or automatically schedule an appointment with the user's healthcare professional. In some examples, the system may send a blink reminder to remind the user to blink regularly or provide eye care reminders to take regular breaks from screen time, adjust display settings to reduce glare and blue light exposure, and avoid prolonged periods of inactivity. In some examples, the notification encourages ceasing use of the electronic device. For example, the system may display a notification on the one or more displays of the electronic device saying “Take a break! Your eyes need rest.”. The notification may be designed to be attention-grabbing and easy to read and may include additional information or recommendations for reducing eye strain. In some examples, to further enhance the effectiveness of the notification, the system may also use haptic feedback and sounds to provide tactile sensation that grabs the user's attention. For example, the device may vibrate slightly to draw the user's attention to the notification or emit a gentle buzzing sound to alert them to take a break. In some examples, the notification combines different aspects such as haptic feedback and sound in a single action, encouraging the user more strongly. In some examples, the different varieties of notifications are displayed separately (e.g., only haptic, or only sound). In some examples, in addition to displaying a message, the system may also display or otherwise convey (e.g., by sound) specific information related to the user, such as blinking metrics or tear film quality. This may help users better understand their eye health and make informed decisions about how to manage their eye health. For example, the system may display a graph showing the typical blinking rate of the user or blinking rate at specific times (e.g., while using specific applications), with suggestions for improving their eye health. In some examples, notifications are customizable based on user preferences, such as tone pitch, vibration intensity, or notification frequency. In some examples, notifications are integrated with other applications to pause notifications and/or minimize screen time during extended use. In some examples, notifications may include schedules reminders for the user to take breaks and perform simple exercises, like rolling the eyes or looking away from the display. In some examples, the notification (or selecting the notification) includes initiating and/or displaying a guided coaching session to influence the user's blink rate, blink duration and/or blink completeness, or any combination thereof. In some examples, dry-eye mitigation may be customizable and tailored to the individual needs and preferences of the user. In some examples, user adherence to dry-eye mitigation is tracked and rewards or other incentives provided for consistent compliance. Furthermore, in some examples, the system may provide additional resources or recommendations for reducing dry-eye condition, eye strain, or other eye conditions and promoting healthy (e.g., pre-determined based on empirical study or based on a baseline for the user of the electronic device) eye habits. By providing these features, the system may help users develop healthy eye habits and reduce their risk of dry-eye and other eye problems. By taking proactive steps to manage their eye health, users can enjoy better overall health and well-being.

In some examples, the system may adjust brightness and contrast to modify blinking behavior of the user, including inducing the user to blink more frequently to prevent or reduce dry-eye conditions by producing more tears. For example, in some examples, the system may increase the brightness to induce the user to blink more frequently in response, thereby producing more tears. Additionally, the system may offer personalized coaching to help the user manage their dry-eye condition, such as providing tips for reducing screen time or adjusting the settings of the one or more displays of the electronic device.

In some examples, the brightness of the one or more displays are increased above a threshold value in order to modify blinking behavior of the user, including inducing the user of the electronic device to blink more frequently. For example, in some examples, threshold factors can include but are not limited to blink frequency, blink completion, spatial properties of tear film temperature regions (e.g., shape, size, etc.), cooling rate, temperatures at various regions of interest, eye redness, blood vessel dilation, and more, or any combination thereof. The threshold values of the determined threshold factors can be predetermined based on the specific attributes of the user of the electronic device, including but not limited to: age, sex, race, ethnicity, location, user history, and other information, or any combination thereof.

In some examples, the device may reduce the fan speeds of the electronic device after determining a blinking condition in order to mitigate dry-eye levels or prevent dry-eye. In some examples, this determination may be done by AI/ML systems that may utilize models that are trained (e.g., supervised learning or unsupervised learning) using various training data. In some examples, the device may increase fan speeds after determining a blinking condition in order to modify blinking behavior of the user, including inducing the user of the electronic device to blink more frequently. In some examples, changing a speed of one or more fans of the electronic device may be done to modify blinking behavior of the user, including inducing blinking of the user of the electronic device, thereby increasing tear production helping to alleviate dry-eye conditions. In some examples, the method may reduce the fan speeds of the electronic device after determining a dry-eye condition to mitigate current dry-eye levels by reducing airflow to the eyes, thereby slowing tear evaporation. In some examples, this determination may be done by AI/ML systems that may utilize models (e.g., implemented as hardware or using hardware to implement software and/or firmware) that are trained (e.g., supervised learning or unsupervised learning) using various training data.

In some examples, warning the user includes a popup notification which includes: providing a notification based on the information, sending a message based on the information, displaying the information, controlling a user interface of an application based on the information, controlling a user interface of a health application based on the information, controlling a focus mode based on the information, setting a reminder based on the information or adding a calendar entry based on the information, or any combination thereof. In some examples, the notification may inform the user to apply a warm compress to the eyes. In some examples, warnings, alerts, and notifications may include information about treatments, therapies, or strategies that address the underlying causes of dry-eye, such as artificial tears, ointments, warm compresses, eye massage, humidity therapy, blinking exercises, avoiding irritants, using anti-inflammatory medications, and more. In some examples, warnings and alerts recommend the user to seek a healthcare professional. In some examples, the method may allow the user to or automatically schedule an appointment with the user's healthcare professional. In some examples, the system may send a blink reminder to remind the user to blink regularly or provide eye care reminders to take regular breaks from screen time, adjust display settings to reduce glare and blue light exposure, and avoid prolonged periods of physical inactivity. In some examples, the notification encourages ceasing use of the electronic device. For example, the system may display a notification on the one or more displays of the electronic device saying “Take a break! Your eyes need rest.”. The notification may be designed to be attention-grabbing and easy to read and may include additional information or recommendations for reducing eye strain. In some examples, to further enhance the effectiveness of the notification, the system may also use haptic feedback and sounds to provide tactile sensation that grabs the user's attention. For example, the device may vibrate slightly to draw the user's attention to the notification or emit a gentle buzzing sound to alert them to take a break. In some examples, the notification combines different aspects such as haptic feedback and sound in a single action, encouraging the user more strongly. In some examples, the different varieties of notifications are displayed separately (e.g., only haptic, or only sound). In some examples, in addition to displaying a message, the system may also display or otherwise convey (e.g., by sound) specific information related to the user, such as blinking metrics or tear film quality. This may help users better understand their eye health and make informed decisions about how to manage their eye health. For example, the system may display a graph showing the typical blinking rate of the user or blinking rate at specific times (e.g., while using specific applications), with suggestions for improving their eye health. In some examples, notifications are customizable based on user preferences, such as tone pitch, vibration intensity, or notification frequency. In some examples, notifications are integrated with other applications to pause notifications and/or minimize screen time during extended use. In some examples, notifications may include schedules reminders for the user to take breaks and perform simple exercises, like rolling the eyes or looking away from the display. In some examples, dry-eye mitigation may be customizable and tailored to the individual needs and preferences of the user. In some examples, user adherence to dry-eye mitigation is tracked and rewards or other incentives provided for consistent compliance. Furthermore, in some examples, the system may provide additional resources or recommendations for reducing dry-eye condition, eye strain, or other eye conditions and promoting healthy eye habits. By providing these features, the system may help users develop healthy eye habits and reduce their risk of dry-eye and other eye problems. By taking proactive steps to manage their eye health, users can enjoy better overall health and well-being.

In some examples, to further enhance the effectiveness of the notification, the system may also use haptic feedback and sounds to provide tactile sensation that grabs the user's attention. For example, the device may vibrate slightly to draw the user's attention to the notification or emit a gentle buzzing sound to alert them to take a break. In some examples, the notification combines different aspects such as haptic feedback and sound in a single action, encouraging the user more strongly. In some examples, the different varieties of notifications are displayed separately (e.g., only haptic, or only sound). In some examples, in addition to displaying a message, the system may also display or otherwise convey (e.g., by sound) specific information related to the user, such as blinking metrics or tear film quality. This may help users better understand their eye health and make informed decisions about how to manage their eye health. For example, the system may display a graph showing the typical blinking rate of the user or blinking rate at specific times (e.g., while using specific applications), with suggestions for improving their eye health. In some examples, notifications are customizable based on user preferences, such as tone pitch, vibration intensity, haptics, or notification frequency. In some examples, notifications are integrated with other applications to pause notifications and/or minimize screen time during extended use. In some examples, notifications may include schedules reminders for the user to take breaks and perform simple exercises, like rolling the eyes or looking away from the display. In some examples, notifications may be customizable and tailored to the individual needs and preferences of the user. In some examples, user adherence to dry-eye mitigation is tracked and rewards or other incentives provided for consistent compliance.

In some examples, the blinking condition is a moist eye condition, and the one or more criteria include a criterion that a frequency of blinks is greater than a threshold. In some examples, the blinking condition may be a neurological condition such as blepharospasm, anxiety, or Tourette syndrome, where the one or more criteria include a criterion that a frequency of blinks is greater than a threshold, inconsistent with the user baseline, and/or irregular compared to a threshold value. In some examples, the blinking condition may be a neurological condition such as Parkinson's disease or Alzheimer's disease, where the one or more criteria include a criterion that a frequency of blinks is less than a threshold. For example, in some examples, threshold factors can include but are not limited to blink frequency, blink completion, spatial properties of tear film temperature regions (e.g., shape, size, etc.), cooling rate, temperatures at various regions of interest, eye redness, blood vessel dilation, and more, or any combination thereof. The threshold values of the determined threshold factors can be predetermined based on the specific attributes of the user of the electronic device, including but not limited to: age, sex, race, ethnicity, location, user history, and other information, or any combination thereof.

In one or more examples of the disclosure, a method performed at an electronic device in communication with one or more displays and one or more thermal image sensors configured to capture thermal imaging data of one or both eyes of a user of the electronic device: receiving the thermal imaging data of the user of the electronic device; extracting one or more features from the thermal imaging data; and in accordance with a determination that one or more criteria are satisfied, the one or more criteria based on the one or more features extracted from the thermal imaging data, determining a dry-eye condition.

In one or more examples, extracting one or more features from the imaging data further includes: classifying a plurality of states of the one or both eyes of the user corresponding to a period of time of the imaging data, wherein the plurality of states includes an open state or a closed state; combining the plurality of states of a first eye and the plurality of states of a second eye into a combined representation for the first eye and second eye; and filtering the combined representation for the first eye and second eye.

In one or more examples, extracting one or more features from the imaging data further includes: classifying a plurality of states of the one or both eyes of the user corresponding to a period of time of the imaging data, wherein the plurality of states includes an open state or a closed state; combining the plurality of states of a first eye and the plurality of states of a second eye into a combined representation for the first eye and second eye; and filtering the combined representation for the first eye and second eye.

In one or more examples, the extracting one or more features from the imaging data further includes: detecting a plurality of peaks in the combined representation after filtering.

In one or more examples, the extracting one or more features from the imaging data further includes: classifying the detected plurality of peaks as a complete blink event, an incomplete blink event for the first eye and the second eye, an incomplete blink event for the first eye, or an incomplete event for the second eye.

In one or more examples, extracting one or more features from the imaging data includes extracting spatial properties of the one or both eyes of the user of the electronic device.

In one or more examples, extracting one or more features from the imaging data includes extracting thermal properties of the one or both eyes of the user of the electronic device.

In one or more examples, extracting one or more features from the imaging data includes extracting at least one of a blinking frequency, a blinking duration, or a blinking completeness of the one or both eyes of the user of the electronic device.

In one or more examples, the one or more criteria include a criterion based on a blinking duration for the one or both eyes.

In one or more examples, the one or more criteria include a criterion based on a blinking completeness for the one or both eyes.

In one or more examples, the one or more criteria include a criterion based on a blinking frequency.

In one or more examples, extracting the one or more features from the imaging data includes applying one or more machine learning models to the imaging data.

In one or more examples, evaluating the one or more criteria includes applying one or more machine learning models to the extracted one or more features.

In one or more examples, the one or more machine learning models include a support vector machine.

In one or more examples, the one or more machine learning models include one or more neural networks.

In one or more examples, extracting one or more features includes detecting a presence of one or more corneal glints of the one or both eyes of the user of the electronic device.

In one or more examples, extracting the one or more features includes detecting a presence of one or more pupils of the one or both eyes of the user of the electronic device.

In one or more examples, the method further comprises: in response to determining a blinking condition based on the one or more features extracted from the imaging data: displaying, via the one or more displays, a notification including instructions regarding blinking.

In one or more examples, the method further comprises: in response to determining a blinking condition based on the one or more features extracted from the imaging data: adjusting a brightness level of the one or more displays.

In one or more examples, adjusting the brightness level of the one or more display include flashing the one or both eyes by transitioning the brightness level above a first threshold brightness level and subsequently transitioning the brightness level below a second threshold brightness level.

In one or more examples, the method further comprises: in response to determining the blinking condition based on the one or more features extracted from the imaging data: changing a speed of one or more fans of the electronic device.

In one or more examples, the method further comprises: in response to determining the blinking condition based on the one or more features extracted from the imaging data: displaying, via the one or more displays, a notification to cease use of the electronic device.

In one or more examples, the electronic device includes one or more audio output devices, and wherein the method further comprises: in response to determining the blinking condition based on the one or more features extracted from the imaging data: providing, via the one or more audio output devices, audio instructions regarding blinking.

In one or more examples, the blinking condition is a dry-eye condition, and the one or more criteria include a criterion that a frequency of incomplete blinks is greater than a threshold.

The present disclosure contemplates that in some examples, the data utilized may include personal information data that uniquely identifies or can be used to contact or locate a specific person. Such personal information data can include demographic data, content consumption activity, location-based data, telephone numbers, email addresses, twitter ID's, home addresses, data or records relating to a user's health or level of fitness (e.g., vital signs measurements, medication information, exercise information), date of birth, or any other identifying or personal information. Specifically, as described herein, one aspect of the present disclosure is tracking a user's biometric data.

The present disclosure recognizes that the use of such personal information data, in the present technology, can be used to the benefit of users. For example, personal information data may be used to display suggested text that changes based on changes in a user's biometric data. For example, the suggested text is updated based on changes to the user's age, height, weight, and/or health history.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates examples in which users selectively block the use of, or access to, personal information data. That is, the present disclosure contemplates that hardware and/or software elements can be provided to prevent or block access to such personal information data. For example, the present technology can be configured to allow users to select to “opt in” or “opt out” of participation in the collection of personal information data during registration for services or anytime thereafter. In another example, users can select not to enable recording of personal information data in a specific application (e.g., first application and/or second application). In addition to providing “opt in” and “opt out” options, the present disclosure contemplates providing notifications relating to the access or use of personal information. For instance, a user may be notified upon initiating collection that their personal information data will be accessed and then reminded again just before personal information data is accessed by the device(s).

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

The foregoing description, for purpose of explanation, has been described with reference to specific examples. However, the illustrative discussions above are not intended to be exhaustive or to limit the disclosure to the precise forms disclosed. Many modifications and variations are possible in view of the above teachings. The examples were chosen and described in order to best explain the principles of the disclosure and its practical applications, to thereby enable others skilled in the art to best use the disclosure and various described examples with various modifications as are suited to the particular use contemplated.

The foregoing description, for purpose of explanation, has been described with reference to specific examples. However, the illustrative discussions above are not intended to be exhaustive or to limit the disclosure to the precise forms disclosed. Many modifications and variations are possible in view of the above teachings. The examples were chosen and described in order to best explain the principles of the disclosure and its practical applications, to thereby enable others skilled in the art to best use the disclosure and various described examples with various modifications as are suited to the particular use contemplated.

Although examples of this disclosure have been fully described with reference to the accompanying drawings, it is to be noted that various changes and modifications will become apparent to those skilled in the art. Such changes and modifications are to be understood as being included within the scope of examples of this disclosure as defined by the appended claims.

What is claimed is:

1. A method comprising:
at an electronic device in communication with one or more displays and one or more image sensors configured to capture imaging data of one or both eyes of a user of the electronic device:
receiving the imaging data of the one or both eyes of the user of the electronic device;
extracting one or more features from the imaging data; and
in accordance with a determination that one or more criteria are satisfied, the one or more criteria based on the one or more features extracted from the imaging data, determining a blinking condition.

2. The method of claim 1, wherein extracting one or more features from the imaging data further includes:
classifying a plurality of states of the one or both eyes of the user corresponding to a period of time of the imaging data, wherein the plurality of states includes an open state or a closed state;
combining the plurality of states of a first eye and the plurality of states of a second eye into a combined representation for the first eye and second eye; and
filtering the combined representation for the first eye and second eye.

3. The method of claim 2, wherein extracting one or more features from the imaging data further includes:
classifying a plurality of peaks as a complete blink event, an incomplete blink event for the first eye and the second eye, an incomplete blink event for the first eye, or an incomplete event for the second eye.

4. The method of claim 3, wherein extracting one or more features from the imaging data includes extracting spatial properties of the one or both eyes of the user of the electronic device.

5. The method of claim 1, wherein extracting one or more features from the imaging data includes extracting thermal properties of the one or both eyes of the user of the electronic device.

6. The method of claim 5, wherein extracting one or more features from the imaging data includes extracting at least one of a blinking frequency, a blinking duration, or a blinking completeness of the one or both eyes of the user of the electronic device.

7. The method of claim 1, wherein the one or more criteria include a criterion based on a blinking duration for the one or both eyes, or
wherein the one or more criteria include a criterion based on a blinking completeness for the one or both eyes, or
wherein the one or more criteria include a criterion based on a blinking frequency.

8. The method of claim 1, wherein the blinking condition is a dry-eye condition, and the one or more criteria include a criterion that a frequency of incomplete blinks is greater than a threshold.

9. An electronic device comprising:
one or more processors;
memory; and
one or more programs, wherein the one or more programs are stored in the memory and configured to be executed by the one or more processors, the one or more programs including instructions for performing a method comprising:
at the electronic device, in communication with one or more displays and one or more image sensors configured to capture imaging data of one or both eyes of a user of the electronic device:
receiving the imaging data of the one or both eyes of the user of the electronic device;
extracting one or more features from the imaging data; and
in accordance with a determination that one or more criteria are satisfied, the one or more criteria based on the one or more features extracted from the imaging data, determining a blinking condition.

10. The electronic device of claim 9, wherein extracting one or more features from the imaging data further includes:
classifying a plurality of states of the one or both eyes of the user corresponding to a period of time of the imaging data, wherein the plurality of states includes an open state or a closed state;
combining the plurality of states of a first eye and the plurality of states of a second eye into a combined representation for the first eye and second eye; and
filtering the combined representation for the first eye and second eye.

11. The electronic device of claim 10, wherein extracting one or more features from the imaging data further includes:
classifying a plurality of peaks as a complete blink event, an incomplete blink event for the first eye and the second eye, an incomplete blink event for the first eye, or an incomplete event for the second eye.

12. The electronic device of claim 11, wherein extracting one or more features from the imaging data includes extracting spatial properties of the one or both eyes of the user of the electronic device.

13. The electronic device of claim 9, wherein extracting one or more features from the imaging data includes extracting thermal properties of the one or both eyes of the user of the electronic device.

14. The electronic device of claim 13, wherein extracting one or more features from the imaging data includes extracting at least one of a blinking frequency, a blinking duration, or a blinking completeness of the one or both eyes of the user of the electronic device.

15. The electronic device of claim 9, wherein the one or more criteria include a criterion based on a blinking duration for the one or both eyes, or
wherein the one or more criteria include a criterion based on a blinking completeness for the one or both eyes, or
wherein the one or more criteria include a criterion based on a blinking frequency.

16. The electronic device of claim 9, wherein the blinking condition is a dry-eye condition, and the one or more criteria include a criterion that a frequency of incomplete blinks is greater than a threshold.

17. A non-transitory computer readable storage medium storing one or more programs, the one or more programs comprising instructions, which when executed by one or more processors of an electronic device, cause the electronic device to perform a method comprising:
at the electronic device, in communication with one or more displays and one or more image sensors configured to capture imaging data of one or both eyes of a user of the electronic device:
receiving the imaging data of the one or both eyes of the user of the electronic device;
extracting one or more features from the imaging data; and
in accordance with a determination that one or more criteria are satisfied, the one or more criteria based on the one or more features extracted from the imaging data, determining a blinking condition.

18. The non-transitory computer readable storage of claim 17, wherein extracting one or more features from the imaging data further includes:
classifying a plurality of states of the one or both eyes of the user corresponding to a period of time of the imaging data, wherein the plurality of states includes an open state or a closed state;
combining the plurality of states of a first eye and the plurality of states of a second eye into a combined representation for the first eye and second eye; and
filtering the combined representation for the first eye and second eye.

19. The non-transitory computer readable storage of claim 18, wherein extracting one or more features from the imaging data further includes:
classifying a plurality of peaks as a complete blink event, an incomplete blink event for the first eye and the second eye, an incomplete blink event for the first eye, or an incomplete event for the second eye.

20. The non-transitory computer readable storage of claim 19, wherein extracting one or more features from the imaging data includes extracting spatial properties of the one or both eyes of the user of the electronic device.

21. The non-transitory computer readable storage of claim 17, wherein extracting one or more features from the imaging data includes extracting thermal properties of the one or both eyes of the user of the electronic device.

22. The non-transitory computer readable storage of claim 21, wherein extracting one or more features from the imaging data includes extracting at least one of a blinking frequency, a blinking duration, or a blinking completeness of the one or both eyes of the user of the electronic device.

23. The non-transitory computer readable storage of claim 17, wherein the one or more criteria include a criterion based on a blinking duration for the one or both eyes, or
wherein the one or more criteria include a criterion based on a blinking completeness for the one or both eyes, or
wherein the one or more criteria include a criterion based on a blinking frequency.

24. The non-transitory computer readable storage of claim 23, the one or more programs further including instructions for:
in response to determining a blinking condition based on the one or more features extracted from the imaging data:
displaying, via the one or more displays, a notification including instructions regarding blinking.

25. The electronic device of claim 17, wherein the blinking condition is a dry-eye condition, and the one or more criteria include a criterion that a frequency of incomplete blinks is greater than a threshold.
